(19) United States
(12) Patent Application Publication
(10) Pub. No.: US 2002/0058787 A1
(43) Pub. Date: May 16, 2002

(54) MHC BINDING PEPTIDE OLIGOMERS AND METHODS OF USE

(76) Inventors: JACK L. STROMINGER, LEXINGTON, MA (US); KIRSTEN FALK, SOMERVILLE, MA (US); OLAF ROTZSCHKE, SOMERVILLE, MA (US)

Correspondence Address: TESTA, HURWITZ & THIBEAULT, LLP, HIGH STREET TOWER, 125 HIGH STREET, BOSTON, MA, 02110, US

(21) Appl. No.: 09/245,487

(22) Filed: Feb. 5, 1999

Related U.S. Application Data
(63) Continuation-in-part of application No. 08/692,167, filed on Aug. 5, 1996, now abandoned.

(30) Foreign Application Priority Data
Aug. 5, 1997 (US) PCT/US97/13885

(51) Int. Cl. C07H 21/02; C07H 21/04; C07K 5/00; C07K 7/00; C07K 16/00; C07K 17/00; A61K 38/00

(52) U.S. Cl. 530/324; 536/23.1

(57) ABSTRACT

Disclosed are oligomers comprising at least two MHC binding peptides joined by a flexible molecular linker. The MHC binding peptides can be MHC class I binding peptides or MHC class II binding peptides. Also disclosed is an oriented cloning method for producing such oligomers. The disclosed oligomers can be used, for example, in connection with methods for specifically activating or inhibiting the activation of CD4⁺ or CD8⁺ T cells. Such methods provide therapeutic approaches for the treatment of tumors, autoimmune disorders, allograft rejection and allergic reactions.

This application is a continuation-in-part of International Application PCT/US97/13885, with an international filing date of Aug. 5, 1997, which claims priority to U.S. application Ser. No. 08/692,167, which was filed on Aug. 5, 1996.

### FIELD OF THE INVENTION

The present invention is directed to the field of immunology and, in particular, to the modulation of immunological responses, the treatment of immunological disorders, allograft rejection, and tumor therapy, as well as diagnostic and in vitro assays based upon immunological responses. In addition, the present invention is directed to reagents and pharmaceutical preparations for use in the foregoing.

### BACKGROUND OF THE INVENTION

Several studies have suggested that dimerization or oligomerization of class II MHC/peptide and of class I MHC/peptide complexes on antigen-presenting cells (APC) together with dimerization or oligomerization of T cell receptors on effector T cells is an essential step in an effective immune response (i.e., that the effective MHC/peptide-TCR complex is a cluster or aggregate). These data include (i) the activation of either T cells or of APC by divalent antibodies that recognize T cell receptors or MHC molecules, but not by monovalent antibodies; (ii) the construction of dominant negative mutants of CD4 that interact with class II restricted TCRs, the effects of which are most readily interpreted by an oligomerization hypothesis; (iii) mutants of class II MHC molecules that suggest that at least two faces of the MHC molecule must be involved in the functional unit; (iv) direct demonstration that dimerization of a class I MHC/peptide complex (but not the monomer) could activate a T cell hybridoma; and (v) structural studies of the class II MHC heterodimer which revealed that this material crystallized as a dimer of the heterodimer as well as similar studies of the Vα domain of a TCR that also crystallized as a dimer, both studies resulting in structural models of how dimerization might be achieved (however, no evidence is available in either case to indicate whether the crystallized dimer is physiologically relevant or a crystallographic artifact). In addition, studies of the peptides bound to class II MHC molecules indicated that in some cases, they were very large and must extend out both ends of the peptide binding groove of the class II MHC molecule, a fact which was definitively established by the crystallographic studies. This structural feature provides the opportunity to link MHC class II binding peptides in order to determine whether aggregation induced by oligomer binding is physiologically relevant.

### SUMMARY OF THE INVENTION

The present invention relates to oligomers comprising at least two MHC binding peptides joined by a flexible molecular linker. The MHC binding peptides can be MHC class I binding peptides or MHC class II binding peptides. In preferred embodiments, the MHC binding peptide oligomers of the present invention comprise at least 4, 8, 16 MHC binding peptides. In some embodiments, the MHC biding peptide oligomers may comprise 32 or more MHC binding peptides.

In the case of MHC class I binding peptide oligomers, the binding peptides are covalently joined C-terminus to C-terminus by synthetic means. The oligomers may, therefore, consist of merely two MHC binding peptides joined by a substantially linear, flexible molecular linker, or of more than two MHC binding peptides joined by a branched, flexible molecular linker. Such flexible molecular linkers may be produced by synthetic chemical techniques.

In the case of MHC class II binding peptide oligomers, the binding peptides may be covalently joined N-terminus to C-terminus by either biosynthetic or synthetic chemical techniques. In the case of biosynthetically produced linkers, the linkers comprise naturally occurring amino acids and may be produced using recombinant DNA vectors described herein. Thus, in another aspect, the invention also relates to an oriented cloning method for producing such oligomers. MHC class II binding peptide oligomers may also be covalently joined N-terminus to N-terminus or C-terminus to C-terminus by synthetic chemical techniques. Such flexible molecular linkers may also be branched or unbranched.

In some embodiments, the flexible molecular linkers of the present invention preferably have backbone lengths of at least about 50-80 Å, and may have backbone lengths of at least about 540 Å or more. When the flexible molecular linkers comprise amino acid residues, they preferably comprise at least about 10-20 amino acid residues, and may comprise at least about 125 amino acid residues or more.

In another aspect, when the flexible molecular linkers are produced by synthetic chemical techniques, they may comprise polymers or copolymers of organic acids, aldehydes, alcohols, thiols, and/or amines; polymers or copolymers of hydroxy-, amino-, and/or di-carboxylic acids (such as polymers or copolymers of glycolic acid, lactic acid, sebacic acid, and/or sarcosine); polymers or copolymers of saturated or unsaturated hydrocarbons (such as polymers or copolymers of ethylene glycol, propylene glycol, or saccharides); polymers or copolymers of naturally and non-naturally occurring amino acids (such as sarcosine and β-alanine) and the like.

The oligomers of the present invention can be used, for example, in connection with methods for specifically activating, or inhibiting activation, of CD4⁺ or CD8⁺ T cells. Such methods represent therapeutic approaches for the treatment of, for example, tumors, autoimmune disorders, allograft rejection, and allergic reactions.

Thus, in one aspect, the present invention provides a method for specifically activating a CD8⁺ T cell to a cell presenting a predetermined antigenic peptide in association with a predetermined MHC class I molecule, by contacting, under physiological conditions, a cell bearing the MHC class I molecule with an MHC binding peptide oligomer comprising at least two agonistic MHC class I binding peptides covalently joined by a flexible molecular linker in which the MHC class I binding peptides correspond to the predetermined antigenic peptide.

In another aspect, the present invention provides a method for specifically inhibiting activation of a CD8⁺ T cell by a cell presenting a predetermined antigenic peptide in association with a predetermined MHC class I molecule, by contacting, under physiological conditions, a cell bearing the MHC class I molecules with an MHC binding peptide oligomer comprising at least two non-agonistic MHC class I binding peptides covalently joined by a flexible molecular linker in which the MHC class I binding peptides correspond to the predetermined antigenic peptide. In this method, the non-agonistic peptide is selected from antagonistic, anergistic, blocking, tolerization-inducing, and apoptosis-inducing peptides.

In another aspect, the present invention provides a method for activating a CD4⁺ T cell toward a cell presenting a predetermined antigenic peptide in association with a predetermined MHC class II molecule, by contacting, under physiological conditions, a cell bearing the MHC class II molecules on its cell surface with an MHC binding peptide oligomer comprising at least two agonistic MHC class II binding peptides covalently joined by a flexible molecular linker in which the MHC class II binding peptides correspond to the predetermined antigenic peptide.

In another aspect, the present invention provides a method for specifically inhibiting activation of a CD4⁺ T cell toward a cell presenting a predetermined antigenic peptide in association with a predetermined MHC class II molecule, by contacting, under physiological conditions, a cell bearing the MHC class II molecules on its cell surface with an MHC binding peptide oligomer comprising at least two non-agonistic MHC class II binding peptides covalently joined by a flexible molecular linker. These peptides may be selected from antagonistic, anergistic, blocking, tolerization-inducing, and apoptosis-inducing peptides.

In another aspect, a method is provided for genetic immunization against a predetermined pathogen by introducing into the cells of a mammal a DNA sequence encoding an MHC binding peptide oligomer comprising at least two immunogenic MHC binding peptides derived from the pathogen and covalently joined by a flexible molecular linker in an expression vector capable of replication and expression in mammalian cells.

In another aspect, the present invention provides a method for eliminating tumor cells from an individual, by contacting under physiological conditions, a cell bearing MHC molecules on its cell surface with an MHC binding peptide oligomer comprising at least two agonistic, tumor-specific MHC binding peptides covalently joined by a flexible molecular linker. In this method the MHC molecules may be MHC class I or MHC class II molecules.

In another aspect, the present invention provides a method for specifically inhibiting activation of a T cell by a predetermined antigenic peptide in association with a predetermined MHC molecule, by contacting, under physiological conditions, a cell bearing the MHC molecule on its cell surface with an MHC binding peptide oligomer comprising at least two of the antigenic peptides covalently joined by a flexible molecular linker at a concentration sufficient for the induction of high zone tolerance. In this method, the T cell may be CD4⁺ or CD8⁺.

In another aspect, the present invention provides a method for producing an immunomodulatory composition, by identifying an MHC binding peptide and preparing an MHC binding peptide oligomer comprising at least two copies of the MHC binding peptide covalently joined by a flexible molecular linker.

In particularly preferred embodiments, the MHC binding peptides of the invention comprise human autoimmunogenic peptides from myelin basic protein (MBP) or proteolipid protein (PLP) for the treatment of multiple sclerosis, AChRα for the treatment of myasthenia gravis, collagen type II or HSP70 proteins for the treatment of rheumatoid arthritis, or glutamic acid decarboxylase for the treatment of insulin-dependent diabetes mellitus (IDDM).

### BRIEF DESCRIPTION OF THE DRAWINGS

FIG. 1 is a diagrammatic representation which illustrates an oriented modular cloning method.

FIG. 2 is a diagrammatic representation which illustrates compatible pairs of restriction sites which are useful in connection with an oriented modular cloning method.

FIG. 3 is a diagrammatic representation which illustrates an amplification step in an oriented modular cloning method.

FIG. 4 is a diagrammatic representation which illustrates the release and further extension of an amplified module in an oriented modular cloning method.

FIG. 5 is a diagrammatic representation of an MHC binding peptide oligomer.

FIG. 6 is a diagrammatic representation of an interface-oligonucleotide.

FIG. 7 is a diagrammatic representation of two oligonucleotide units used in the construction of an MHC binding peptide oligomer.

### DETAILED DESCRIPTION OF THE INVENTION

The present invention relates to compositions and methods for modulating the immune response in an individual. In particular, the invention is based, in part, upon the finding that oligomers comprising class I or class II MHC binding peptides, joined by one or more flexible molecular linkers, are characterized by a significantly increased ability to modulate an immune response, when compared to monomers of MHC binding peptides.

Definitions.

In order to more clearly and concisely describe and point out the subject matter of the claimed invention, the following definitions are provided for specific terms which are used in the following description and the claims appended hereto.

As used herein, the term or “MHC molecule” means an MHC class I molecule and/or an MHC class II molecule.

As used herein, the term “MHC class I” or “class I” refers to the human Major Histocompatibility Complex class I molecules, binding peptides or genes. The human MHC region, also referred to as HLA, is found on chromosome six and includes the class I region and the class II region. Within the MHC class I region are found the HLA-A, HLA-B or HLA-C subregions for class I α chain genes. The human gene for β₂-microglobulin is located outside the MHC complex on a separate chromosome. As used herein, the term “MHC class I molecule” means a complex of an MHC class I α chain and a β₂-microglobulin chain. MHC class I molecules normally bind peptides which are generated in the cytosol and transported to the endoplasmic reticulum. After binding these peptides, the class I MHC-peptide complex is presented on the cell surface where it may be recognized by T cells. The majority of bound peptides have a length of 8-10 amino acids, although they may be as long 16 or as short as 2 (Udaka et al., (1993) Proc. Natl. Acad. of Sci. (USA) 90:11272-11276). See, generally, Roitt et al., eds. Immunology (1989) Gower Medical Publishing, London.

As used herein, the term “MHC class II” or “class II” refers to the human Major Histocompatibility Complex class II molecules, binding peptides or genes. The human MHC region, also referred to as HLA, is found on chromosome six and includes the class I region and the class II region. Within the MHC class II region are found the DP, DQ and DR subregions for class II α chain and β chain genes (i.e., DPα, DPβ, DQα, DQβ, DRα, and DRβ). As used herein, the term “MHC class II molecule” means a complex of an MHC class II α chain and an MHC class II β chain. MHC class II molecules normally bind peptides in an intracellular processing compartment and present these peptides on the surface of antigen presenting cells to T cells. The majority of bound peptides have a length of 13-18 amino acids but it is the peptide side chains of an approximately 9 amino acid core segment that occupy pockets of the MHC class II binding cleft and determine the specificity of binding (Brown et al., (1993) Nature 364:33-39; Stem et al., (1994) Nature 368:215-221). See, generally, Roitt et al., eds. Immunology (1989) Gower Medical Publishing, London.

As used herein, the term “MHC binding peptide” or “binding peptide” means an MHC class I binding peptide and/or an MHC class II binding peptide.

As used herein, the term “MHC class I binding peptide” means a polypeptide which is capable of selectively binding within the cleft formed by the α and β₂-microglobulin chains of a specified MHC class I molecule to form an MHC class I-peptide antigen complex. An MHC class I binding peptide may be a processed self- or non-self peptide or may be a synthetic peptide. For class I MHC molecules, the peptides are typically 8-10 amino acids in length, although they may be as long 16 or as short as 2. In particular, as the oligomers of the present invention comprise MHC binding peptides joined by flexible molecular linkers, the MHC binding portions of the oligomers may comprise only the approximately 8-10 amino acid core segments that occupy the MHC class binding clefts.

As used herein, the term “MHC class II binding peptide” means a polypeptide which is capable of selectively binding within the cleft formed by the α and β chains of a specified MHC class II molecule to form an MHC class II-peptide antigen complex. An MHC class II binding peptide may be a processed self- or non-self peptide or may be a synthetic peptide. For class II MHC molecules, the peptides are typically 10-25 amino acids in length, and more typically 13-18 residues in length, although longer and shorter ones may bind effectively. In particular, as the oligomers of the present invention comprise MHC binding peptides joined by flexible molecular linkers, the MHC binding portions of the oligomers may comprise only the approximately 9 amino acid core segments that occupy the MHC class binding clefts.

As used herein with respect to MHC binding peptides, an “oligomer” means a molecule comprising at least two MHC binding peptides which are covalently joined, optionally by a flexible molecular linker. Preferably, the oligomers of the present invention comprise at least 2-4 MHC binding peptides, more preferably at least 4-16 MHC binding peptides, and most preferably 4-32 MHC binding peptides, which are covalently joined. Optionally, but preferably, the MHC binding peptides are covalently joined by flexible molecular linkers interposed between the MHC binding peptides.

As used herein, the term “flexible molecular linker” or “linker” means a chemical moiety which covalently joins two MHC binding peptides, having a backbone of chemical bonds forming a continuous connection between adjacent the MHC binding peptides, and having a plurality of freely rotating bonds along that backbone. In preferred embodiments, the flexible molecular linkers of the invention have a backbone length (i.e., the sum of the bond lengths forming a continuous connection between the MHC binding peptides) of at least about 50-60 Å. Preferably, a flexible molecular linker comprises a plurality of amino acid residues but this need not be the case.

As used herein, the term “selectively binding” means capable of binding in the electroand and stereospecific manner of an antibody to antigen or ligand to receptor. With respect to an MHC binding peptide, selective binding entails the non-covalent binding of specific side chains of the peptide within the binding pockets present in the MHC binding cleft in order to form an MHC-peptide complex (see, e.g., Brown et al., (1993) Nature 364:33-39; Stem et al., (1994) Nature 368:215-221; Stem and Wiley (1992) Cell 68: 465-477).

As used herein, the term “substantially pure” means, with respect to the MHC binding peptides and the oligomers of the invention, that these molecules are essentially free of other substances to an extent practical and appropriate for their intended use. In particular, the molecules are sufficiently pure and are sufficiently free from other biological or immunogenic constituents so as to be useful in, for example, modulating a specific immune response or producing pharmaceutical preparations. A substantially pure preparation of the oligomers of the invention need not be absolutely free of all other proteins or molecules and, for purposes of administration, may be relatively dilute. Thus, for example, the oligomers may be in a solution including various buffers, excipients, or adjuvants. One of ordinary skill in the art may produce such substantially pure preparations by application or serial application of well-known methods including, but not limited to, HPLC, affinity chromatography or electrophoretic separation. As the substantially pure preparations of the invention may also comprise various biologically active or inactive ingredients (e.g., water, buffers, excipients, adjuvants), the percentage by weight of the MHC binding peptides or oligomers of the invention may be reduced in such a preparation.

MHC Molecules and Binding Peptides

MHC class I and class II molecules are cell surface glycoproteins which, although different in some ways, share many common structural features. One of these structural features is an extracellular antigen binding cleft. Peptide fragments are bound in this antigen binding cleft during the steps which comprise intracellular antigen processing. MHC molecules, charged with antigenic peptide, are transported to the cell surface where the bound peptides are displayed to T cells. T cells contain specialized receptors which recognize antigenic peptide fragments in association with an MHC molecule. In response to such recognition, T cells can be activated, thereby stimulating a signaling cascade forming the basis of the immune response.

At the most fundamental level, the immune system functions to rid the body of pathogens (e.g., viruses, bacteria, pathogenic fungi and eukaryotic parasites). The role of T cells in the immune response depends upon their ability to recognize cells harboring such pathogens. Structural and functional distinctions between the two classes of MHC molecules enables them to bind to, and present, a wide range of antigenic peptides derived from such pathogens in each of the two major intracellular compartments of cells—the cytosol and internal vesicles. This process of antigenic peptide binding to, and presentation by, MHC molecules has been referred to as peptide “charging” or “loading” of the MHC molecule.

MHC class I molecules, as contrasted with MHC class II molecules, are charged with peptides derived from proteins which are produced primarily in the cytosol of a cell. In cells infected by viruses, for example, viral proteins are produced in the cytosol. Fragments of viral proteins are transported into the endoplasmic reticulum where they are bound by MHC class I molecules. These charged MHC class I molecules are then transported to the cell surface.

MHC class II molecules, on the other hand, are primarily charged with peptides derived from proteins processed within intracellular membrane-bound vesicles. These intracellular membrane-bound vesicles contain, for example, proteins engulfed by macrophages or internalized by B cells.

These two classes of MHC molecules, in their charged form on the surface of a cell, are recognized by different functional classes of T cells. For example, charged MHC class I molecules are recognized by CD8+ cytotoxic T cells. Charged MHC class II molecules are recognized by, for example, CD4⁺ helper T cells.

The structures of both MHC class I and MHC class II molecules have been determined by X-ray crystallography. MHC class I molecules consist of two polypeptide chains, an α chain encoded in the MHC, and a smaller non-covalently associated chain, β-2 microglobulin, which is not encoded in the MHC. The molecule has four domains, three formed from the MHC-encoded a chain, and one from β-2 microglobulin. The α₁ and α₂ domains pair to generate a cleft on the surface of the molecule that is the site of antigen binding. When antigenic peptide is bound in the cleft of an MHC class I molecule, the N-terminus of the antigenic peptide is substantially buried (and therefore inaccessible) within the cleft. The C-terminus of the antigenic peptide, on the other hand, is more accessible than the N-terminus.

MHC class II molecules consist of a non-covalent complex of two chains, α and β, both of which span the membrane. The crystal structure of the MHC class II molecule reveals that it has a folded structure very similar to that of the MHC class I molecule. The most significant differences in the folded structure of the two molecules lie at the ends of the peptide binding cleft, which are substantially more open in MHC class II molecules. The main consequence of these differences is that the ends of a peptide bound to an MHC class I molecule are more embedded, whereas the ends of peptides bound to MHC class II molecules are more exposed.

The amino acid sequences of many MHC class I and class II binding peptides are currently known, and others can be determined through routine experimentation well known to those skilled in the art (see, e.g., Rammensee et al., (1995) Immunogenetics 41: 178-228). For example, if the peptide antigen has been isolated it is possible to identify its sequence by techniques such as Edman degradation (Nelson et al., (1992) Proc. Natl. Acad. Sci. USA 89: 7380-7383) and mass spectrometric methods (see, e.g., Cox et al., (1994) Science 264: 716-719). In addition, MHC binding peptides can be identified by scanning the sequence of a protein of interest with the respective consensus-motif of the restricting MHC-molecule (see, e.g., WO96/27387). In general, consensus-motifs of MHC-ligands are allele-specific (i.e., the motif of peptides bound, for example, to HLA-A2.1 is different from the motif of peptides which bind to HLA-B2701). Such motifs summarize invariant features contained within such peptides including, for example, length and position of the invariant amino acid positions. Consensus motifs have been identified for the ligands of MHC class I and class II and methods for the identification of such motifs have been described. These include, for example, pool sequencing (Falk et al., (1991) Nature 351: 290-296; Falk et al., (1994) Immunogenetics 39: 230-242) as well as the use of phage display libraries (e.g., Hammer et al., (1992) J. Exp. Med. 179: 1007-1013); selected motifs are specifically disclosed by Rammensee et al., (1995) Immunogenetics 41: 178-228.

In preferred embodiments the MHC binding peptides are selected from the group consisting of (1) residues 57-75, residues 81-99, residues 82-100, residues 83-97, residues 83-99, residues 84-102, residues 87-99, residues 87-106, residues 131-145, residues 131-159, residues 139-153, residues 143-168, residues 148 162, residues 149 162, and residues 154-172 of myelin basic protein (MBP); (2) residues 40-60, residues 89-106, and residues 139-151, of proteolipid protein (PLP); (3) residues 48-67, residues 55-63, residues 144-163, residues 146-162, residues 195-212, residues 214-234, residues 259-271, residues 311-318, and residues 387-405, of AChRα; (4) residues 258-260, residues 258-270, residues 259-273, residues 260-267, residues 261-273, residues 262-270 and residues 263 270 of collagen type II; (5) residues 150-169, residues 221-240 and residues 261-280 of the of HSP70 protein of M. tuberculum; and (6) residues 78-97, residues 202-211, residues 217-236, and residues 247-279 of glutamic acid decarboxylase.

Oligomers of MHC Binding Peptides

As discussed above, several studies have suggested that dimerization or oligomerization (also referred to as “clustering”) of MHC class I-peptide and of MHC class II-peptide antigen complexes, together with dimerization or oligomerization of T cell receptors on effector T cells, appears to be an essential step in an effective immune response. The subject invention is based, in part, on the discovery that oligomers comprising MHC binding peptides, joined by one or more flexible molecular linkers, are characterized by a significantly increased ability to modulate an immune response through oligomer-induced clustering.

The term MHC binding peptides, as noted above, refers to peptides which bind specifically within the antigen binding cleft of MHC class I or MHC class II molecules. These can be epitopes (i.e., peptide sequences presented by an antigen presenting cell to a T cell with functional consequences with regard to T cell activity), or peptide sequences which bind to MHC molecules and stimulate APC activity (e.g., lymphokine secretion) without T cell involvement, or peptide sequences which bind to an MHC molecule and block the MHC molecule from binding other epitopes (i.e., without direct functional consequences with respect to T cell activity).

The compositions of the present invention are oligomers comprising two or more MHC binding peptides optionally joined by one or more flexible molecular linkers. For most applications, identical MHC binding peptides will be joined by a flexible molecular linker. However, it is also possible to design an oligomer which contains more than one type of MHC binding peptide of the same class (i.e., two or more MHC class I binding peptides, or two or more MHC class II binding peptides). An oligomer containing MHC binding peptides of different identities would be useful, for example, when one of the MHC binding peptides is known to bind MHC molecules with low affinity. In this case, linking the low affinity binding peptide to a peptide which binds MHC molecules with higher affinity functions to increase the local concentration of the low affinity binder at the surface of an antigen presenting cell. In addition, two or more non-identical MHC binding peptides which share a common MHC binding motif may be linked. Although the primary sequence of such peptides may be non-identical, the fact that they share a common MHC binding motif may be particularly useful, for example, in the depletion of specific T cell subsets which may be associated with an autoimmune disorder.

As discussed above, and reported in the prior art, the geometry of the peptide binding cleft of MHC class II molecules is relatively open at the point where the N- and C-termini of a bound antigenic peptide protrude from the MHC class II molecule. Thus, the N- and C-termini of antigenic peptides bound by MHC class II molecules are accessible, and modification of the N- and/or C-termini of MHC class II binding peptides generally does not function to sterically hinder the specific binding of such peptides by MHC class II molecules.

Because their N- and C-termini are accessible, the preferred method for linking MHC class II binding peptides is N- terminus to C-terminus. This method offers a significant advantage in light of the fact that such linkages can be produced biosynthetically, in vivo. For biosynthetic production, the flexible molecular linker which joins the MHC class II binding peptides will necessarily comprise naturally occurring (i.e., L-isomer) amino acids. DNA encoding such oligomers is synthesized and cloned in a DNA expression vector using conventional techniques (see below). Although biosynthetic methods are the preferred method for linking MHC class II binding peptides N-terminus to C-terminus, any conventional chemical synthetic technique can be employed (see below).

In addition to oligomers of MHC class II binding peptides linked N-terminus to C-terminus, such oligomers can be produced by linking two peptides N-terminus to N-terminus, or two peptides C-terminus to C-terminus. Alternatively, mixed oligomers can be produced linking, for example, two peptides joined C-terminus to C-terminus, to the N-terminus of a third MHC binding peptide. This type of mixed oligomer strategy can be used to generated high molecular weight oligomers. With respect to oligomers of MHC class II binding peptides linked C-terminus to C-terminus or N-terminus to N-terminus, biosynthetic techniques are not an option. Such oligomers must be linked by conventional synthetic techniques as described below.

Oligomers of MHC class I binding peptides must be linked C-terminus to C-terminus. This requirement stems from the fact that, as discussed above, the N-terminus of an antigenic peptide bound in the antigen binding cleft of an MHC class I molecule is buried, and presumed inaccessible. Thus, it is presumed that any linker added at the N-terminus to an MHC class I binding peptide would interfere with its ability to specifically bind MHC class I molecules. The X-ray data does reveal, however, that the C-terminal amino acid residue of antigenic peptides bound in the antigen binding cleft of MHC class I molecules is more accessible. Thus, linking two MHC class I molecules C-terminus to C-terminus through a flexible molecular linker will not substantially inhibit the ability of the MHC class I binding peptides to specifically bind to MHC class I molecules. In one experiment, for example, a glycine residue was added to the C-terminus of a nonameric peptide epitope yielding a decamer. The decamer binds in the cleft with the C-terminal carboxyl residue oriented outside the peptide binding cleft (Collins et al., (1995) Nature 371: 626-629) and, therefore, the C-terminus is accessible for the addition of a flexible molecular linker. Therefore, using either glycine residues or other chemical groups at the ends of a flexible molecular linker, dimers of MHC class I binding peptides may be produced. In addition, by using branched linkers, higher oligomers (e.g., oligomers with 4-32 MHC binding peptides) may be produced by routine chemical synthesis and can be readily tested by any of the functional assays described in the examples below.

For biosynthetic synthesis of oligomers in which the MHC binding peptides are joined N-terminus to C-terminus by flexible polypeptide linkers, nucleic acids may be produced which will encode the oligomers. Briefly, DNA sequences which encode the MHC binding peptides are designed by reference to the genetic code. These DNA sequences are joined to DNA sequences encoding flexible polypeptide linker sequences such that the linker sequences are interspersed with the MHC binding peptide sequences. The length of the flexible molecular linker can be variable, and in oligomers comprising 3 or more MHC class II binding peptides (which are spaced by two or more flexible molecular linkers) the flexible molecular linkers need not be of uniform length or composition. The flexible molecular linkers are substantially linear and preferably inert, hydrophilic and noncleavable by proteases. The flexible molecular linkers are also preferably designed to lack secondary structure under physiological conditions. Thus, for example, the polypeptide linker sequences are preferably composed of a plurality of residues selected from the group consisting of glycine, serine, threonine, cysteine, asparagine, glutamine, and proline. Particularly preferred are polypeptide linkers consisting essentially of glycine, alanine and proline residues. Polypeptide linkers including the larger, aromatic residues may also be included, although they are less preferred because they may cause steric hindrance. Similarly, the charged amino acids may be included, but they are less preferred because they may interact to form secondary structures, and the nonpolar amino acids may be included, but they are less preferred because they may decrease solubility. The recitation of these preferred characteristics, however, are not intended to be exclusive. While it is believed that the characteristics specified would promote optimum activity, flexible polypeptide linkers which do not satisfy the preferred criteria may prove to be at least as effective. Whether this is the case can be determined by routine experimentation given the teaching of the present specification.

Thus, a DNA sequence encoding an oligomer of the type described above can be inserted into an expression vector. The expression vector encoding the oligomer of the invention is then introduced into an appropriate cell type where it is expressed. Prokaryotic cells (e.g., E. coli) represent a preferred host system although the oligomers can be expressed efficiently in eukaryotic systems given appropriate vector selection. Oligomers which are synthesized in such host cells may then be isolated by conventional techniques and formulated for administration to a mammalian, preferably human, subject. Alternatively, as discussed below in connection with therapeutic methods, the DNA encoding an oligomer of the present invention can be inserted into a eukaryotic vector suitable for use in connection with genetic immunization.

For chemical synthesis of flexible molecular linkers, one of skill in the art of organic synthesis may design a wide variety of linkers which satisfy the requirements discussed above. Thus, depending upon the nature of the termini to be joined (i.e., N- and/or C-termini), appropriate end groups are chosen for the linker such that the linker may be joined to the chosen termini of the MHC binding peptides (e.g., using a naturally occurring amino acid, D-isomer amino acid, or modified amino acid, such as sarcosine or β-alanine, at one or both ends). Preferred flexible molecular linkers include polymers or copolymers of organic acids, aldehydes, alcohols, thiols, amines and the like. For example, polymers or copolymers of hydroxy-, amino-, or di-carboxylic acids, such as glycolic acid, lactic acid, sebacic acid, or sarcosine may be employed. Alternatively, polymers or copolymers of saturated or unsaturated hydrocarbons such as ethylene glycol, propylene glycol, saccharides, and the like may be employed. One example of such s flexible molecular linker is polyethylene glycol (with or without, e.g., β-alanine at the ends), as described below in Example 2. Other examples include polymers or copolymers of non-naturally occurring amino acids (including, for example, D-isomers). Certain non-naturally occurring amino acids have characteristics which would be predicted to be advantageous in connection with the oligomers of the present invention. For example, N-methyl glycine (sarcosine) would be predicted to minimize hydrogen bonding and secondary structure formation while exhibiting favorable solubility characteristics and, therefore, a polysarcosine linker (with or without, e.g., lysine at the ends) may be employed, as described below in Example 2. These and many other flexible molecular linkers may be readily employed by one of ordinary skill in the art using traditional techniques of chemical synthesis.

As a general matter, if an MHC binding peptide dimer is employed, it is preferred that the flexible molecular linker has a length sufficiently long to at least span the distance between the termini of the MHC binding peptides when they are bound in the MHC binding clefts of adjacent or clustered MHC molecules. Preferably, however, they are longer, so as to bind to non-adjacent or non-clustered MHC molecules and promote their clustering. Thus, for example, as shown in Example 2, a polyethylene glycol (PEG) linker was employed with an end-to-end length of approximately 30-40 Å. Such a linker will have a backbone length (i.e., the sum of the bond lengths forming a continuous connection between adjacent MHC binding peptides) of approximately 60-80 Å (see, e.g., CRC Handbook of Chemistry and Physics, 76th Edition, CRC Press (1995), for bond lengths; the backbone length is longer than the end-to-end length because the chemical bonds are not arranged linearly). Similarly, in Example 1, polypeptide linkers comprising 12-13 amino acid residues were employed. Such flexible molecular linkers will have backbone lengths of approximately 50-60 Å. Thus, for embodiments in which the MHC binding peptide oligomer is a dimer, it is preferred that the flexible molecular linker have a backbone length of at least 50-80 Å, and preferably more.

As a general matter for higher oligomers, as noted in Example 1, it appears that not all of the MHC binding peptide subunits in the oligomer will actually bind an MHC molecule. Rather, as shown below, it appears that, using MHC binding peptides of 13 amino acid residues each, with spacers of 12 amino acids each, it appears that an additional MHC molecule is bound for each 5-mer or 6-mer in the oligomer. As noted below, this translates to a distance of approximately 125-150 amino acid residues between bound MHC molecules, or a backbone length of approximately 540-645 Å. In this example, the MHC binding peptide units which failed to bind MHC molecules may be regarded, in one sense, as merely contributing to the length of the flexible molecular linker and, therefore, for some embodiments, flexible molecular linkers of 540-645 Å or longer may be preferred. At the same time, the use of higher oligomers (e.g., 4-mers to 32-mers), by providing more binding sites, increases the probability of binding MHC molecules. Thus, as an arbitrary example, assuming a 16-mer which binds 3 MHC molecules separated by exactly 6 monomer units, the MHC molecules may be bound at relative monomer positions of 1, 7 and 13; 2, 8, and 14; 3, 9, and 15; or 4, 10 and 16. Therefore, it is unpredictable which monomer units will bind MHC molecules, and it is preferred that the oligomers include a high density of MHC binding peptide monomers even if some remain unbound. Furthermore, as some of the MHC binding peptide units may serve, in effect, as flexible molecular linkers, the backbone length of these peptides may be included when calculating the backbone length of the flexible molecular linker between any two non-adjacent MHC binding peptide units.

Finally, for higher oligomers (e.g., 4-mers to 32-mers), the flexible molecular backbone may be either reduced or eliminated because, for non-adjacent MHC binding peptide units, the intervening MHC binding units may serve as flexible molecular linkers. Alternatively phrased, for higher oligomers, internal MHC binding peptide units may be regarded as flexible molecular linkers for the more distal MHC binding peptide units which they join.

Thus, in preferred embodiments, the MHC binding peptide oligomers of the present invention comprise MHC binding peptides joined by flexible molecular linkers having backbone lengths of at least 50-60 Å, or 60-80 Å, when only dimers are employed, and having backbone lengths of anywhere from 50-60 Å to 540-645 Å, when 4-mers or higher oligomers are employed. In addition, in preferred embodiments, the oligomers are 4-mers to 32-mers, more preferably 12-mers to 24-mers, and most preferably about 16-mers, in which the MHC binding peptides are joined by flexible molecular linkers having backbones of about 50-60 Å or 60-80 Å. In particularly preferred embodiments, the flexible molecular linkers are polypeptide linkers comprising anywhere from 10-20 to 125-150 amino acid residues.

Methods Employing Oligomers of MHC Binding Peptides

The oligomers of the present invention can be employed in a variety of therapeutic contexts to specifically activate or inhibit CD8⁺ or CD4⁺ T cells. Methods for activating such T cells are generally used in connection with, for example, vaccination to induce an immune response. Activation of T cells is mediated by an agonistic MHC binding peptide. In addition to vaccination against common pathogens, such a method can also be used to stimulate a T cell response directed toward an antigen which could be characterized as weakly immunogenic. For example, tumor-specific antigens would fall within this category (Boon et al., (1994) Ann. Rev. Immunol. 12: 337-365; Topalian et al., (1996) J. Exp. Med. 183: 1965-1971). Tumor cells are not, in many instances, efficiently eliminated from the body by the immune system. The stimulation of the T cell reactivity associated with the oligomers of the present invention offers a method for inducing a substantially more effective response. As discussed previously, antigenic peptides which bind specifically to MHC class I or MHC class II molecules are either known, or can be easily determined experimentally.

To stimulate T cell response against a particular agonistic antigenic peptide, an oligomer of the type described above is produced, and administered to an individual. A subset of the MHC molecules normally present on the surface of cells are known to be empty (i.e., the antigen binding cleft is not charged with an MHC binding peptide). In addition, displacement of weaker binding peptides by more strongly binding peptides has also been demonstrated experimentally. Thus, the oligomers of the present invention, when contacted with MHC molecules on the surface of cells in vivo or in vitro, may bind to the MHC molecules either by binding to empty MHC molecules or by displacing less strongly binding peptides from already charged MHC molecules. T cells (either CD8⁺ or CD4⁺) recognize the bound antigen in connection with an appropriate MHC molecule. The resulting clustered complex, as demonstrated in the experiments described below, causes superactivation (i.e., the amount of antigen required to trigger a T cell response is orders of magnitude lower compared to the response triggered in otherwise identical experiments using unlinked antigen).

In addition to methods for stimulating a T cell response, the methods of the present invention can be used to inhibit an immune response (i.e., immunosuppression). Such methods would generally be indicated in connection with autoimmune diseases, allograft rejection, or allergic states.

Autoimmune diseases are often correlated with the expression of one or a limited number of allelic forms of MHC molecules. In most cases, this correlation is found with MHC class II molecules. Examples of this class include, for example, rheumatoid arthritis, multiple sclerosis and insulin-dependent diabetes mellitus (IDDM).

Rheumatoid arthritis has been correlated with the expression of a subtype of HLA-DR1 or -DR4 (e.g., HLA-DR0401). The autoantigen responsible for the disease phenotype is not known, but collagen type II and HSP70 have been implicated as potential sources (reviewed by Feldman et al., (1996) Cell 85: 307-310).

Multiple sclerosis (MS) is the most common disease involving the nervous system. It is correlated to the expression of HLA-DR2. The autoantigens are provided by at least two proteins, myelin basic protein (MBP) and proteolipid protein (PLP), both major constituents of the myelin sheath. Immunodominant T cell epitopes have been described for MBP (Ota et al., (1990) Nature 346: 183-187; Allegretta et al., (1990) Science 247: 718-721); and PLP (Pelfrey et al., (1993) J. Neuroimmunol. 46: 33-42). The correlate of this autoimmune disease in the mouse model system is experimental allergic encephalomyelitis (EAE). Recent publications have reported that EAE can be treated by application of altered peptide analogues of MBP (Brocke et al., (1996) Nature 379: 343-346) and PLP (Nicholson et al., (1995) Immunity 3(4): 397-405).

Susceptibility to insulin-dependent diabetes mellitus (IDDM) or type I diabetes is most strongly correlated with the expression of several HLA-DQ alleles. T cell responses have been reported which are directed against autoantigens, for example, deriving from glutamic acid decarboxylase (Atkinson et al., (1994) J. Clin. Invest. 94: 2125-2129). The nonobese diabetic (NOD) mouse represent a commonly used animal model system.

In addition to the autoimmune diseases associated with the expression of a particular MHC class II allele, a few autoimmune diseases are presently known in which a linkage to the expression of a particular MHC class I molecule has been determined. For example, ankylosing spondylitis correlates with HLA-B27 (Benjamin and Parkham, (1990) P. Immunol. Today 11: 137-142).

Oligomers comprising non-agonistic MHC binding peptides are used in connection with this immunosuppression embodiment. Non-agonistic peptides are peptides which, following binding by the appropriate MHC molecule, do not result in T cell activation. A number of types of non-agonistic peptides have been described including, for example, antagonistic, anergistic, blocking, tolerization-inducing, and apoptosis-inducing peptides. Such activities are well characterized in the literature.

Non-agonistic peptides which inhibit a T cell response (e.g., anergistically, antagonistically, etc.) can be generated through routine experimentation by modification of known agonistic peptides, followed by appropriate testing (Sette et al., (1994) Ann. Rev. Immunol. 12: 413-431; Sloan-Lancaster, (1996) Ann. Rev. Immunol. 14: 129). In particular, modification of one or more outwardly pointing T cell contact residues (i.e., outward with respect to the peptide binding cleft of the MHC molecule) in an MHC binding peptide can convert an agonistic peptide to a non-agonistic peptide. For example, residues P1, P2, P3, P5, P7 and P8 in the hemagglutinin peptide HA 306-318 (which binds to an HLA-DR1 molecule) represent the outwardly pointing T cell contact residues. Modification of any of these residues can result in peptides that are antagonizing or anergizing (Sloan-Lancaster and Allen, (1996) Ann. Rev. of Immunol. 14: 129). Natural amino acids are generally used for substitution but in synthetic approaches other organic molecules might be used, including derivatized amino acids.

In the therapeutic methods discussed above, oligomers of the invention are produced (either in a host cell or in vitro), isolated and administered to an individual. However, recent advances have led to the development of a technique termed genetic immunization (Ulmer et al., (1993) Science 259: 1745). In experiments conducted in a murine system, a naked DNA plasmid containing the gene for hemagglutinin was injected directly into muscle. Influenza hemagglutinin contains both B- and T-cell epitopes. In response to this injection, a flu-specific immune response consisting of both antibody and cytotoxic T cells was stimulated. This response can be enhanced by coinjecting a plasmid encoding a cytokine. It is presumed that the plasmids DNAs are expressed by some of the cells in the muscle tissue into which it was injected, thereby stimulating the specific immune response.

DNA encoding the oligomers of the present invention, inserted in an appropriate expression vector, could be used in such a genetic immunization protocol. One of skill in the art will recognize that certain intracellular signal sequences may be required in order to target an oligomer synthesized within a cell, into an appropriate cellular location for binding with MHC class I or class II molecules.

In addition to the methods described above, the compositions of the present invention appear to be useful in connection with the induction of high zone tolerance. High zone tolerance is a phenomenon wherein the application of high concentrations of soluble, otherwise agonistic, MHC binding peptides leads to tolerization rather than stimulation of an immune response (see, e.g., Weigle, (1973) Adv. Immunol. 16: 61-122; Liblau et al., (1996) Proc. Natl. Acad. Sci. USA 93: 3031-3036). As shown in the experiments described below, the use of an oligomer of the present invention functions to shift the dose response curve in a standard, T cell assay with an established T cell line and also in an in vitro priming assay using peripheral T cells isolated from blood. Given these results, it appears that oligomer-mediated clustering can induce high zone tolerance at relatively low concentrations.

The ability to induce high zone tolerance at relatively low MHC binding peptide concentration offers therapeutic advantages. Previous reports of high zone tolerance induction have required the administration of peptide concentrations at such high concentrations that practical application in a therapeutic context was not possible. However, using the oligomers of the present invention, much lower concentrations are required. Thus, for example, an oligomer comprising agonistic peptides associated with autoimmune disease (e.g., MBP 84-102 for multiple sclerosis; or AChR α144-163 for myasthenia gravis) can be administered in a tolerizing dosage to a individual afflicted with such a disorder. Such a dosage can be determined empirically using, for example, mixed PBMCs in vitro and non-human mammalian animals in vivo.

### EXAMPLES

### Example 1

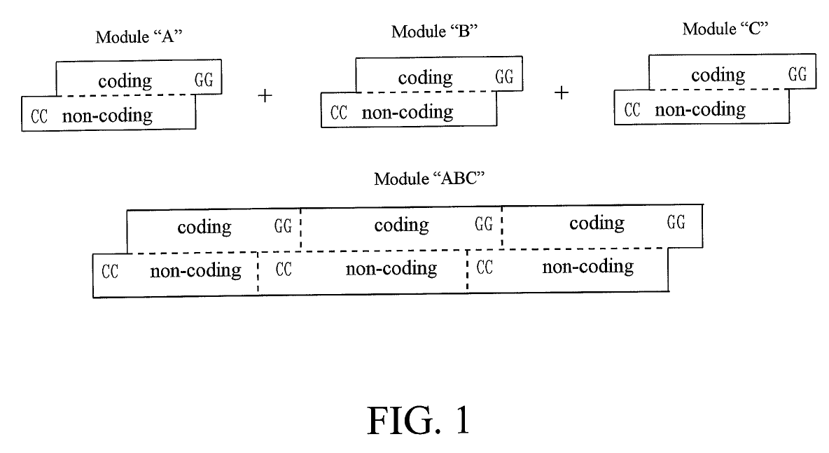

Polypeptide oligomers of the class II MHC-restricted T cell epitope HA306-318 separated by spacers or linkers of 12 amino acids (G-P-G-G)₃ were constructed using a novel modular cloning method. This methodology is particularly designed for the generation of large oligomers consisting of a large number of relatively small repetitive oligonucleotide units. This strategy can also be applied to oligonucleotides that are to be constructed so that additional non-repetitive units can be connected to or incorporated into a construct. Classical cloning methods are based on the use of identical or compatible restriction enzyme sites. These sites have to be located such that compatible ends can be generated to form a linkage between two different DNAs. These restriction enzyme sequences are always part of the coding sequence and can interfere with the desired amino acid sequence encoded by the oligomer. Furthermore, to connect identical units, it is preferred that they carry compatible overhangs on both sides. However, most standard restriction enzymes require palindromic cutting sites and produce either blunt-ends or compatible but identical overhangs on both strands, so that the ligation can occur in both orientations (i.e. coding strand—coding strand or coding strand—non-coding strand). Furthermore, if identical restriction sites are used at both ends of the individual unit, the restriction site is reconstituted at the junction of the ligated product as well as on its ends. A release of the cloned product with this enzyme (for example, for further extensions) is therefore not possible. The strategy introduced here avoids these complications, since the modular elements do not require the presence of recognition sites. The modular elements used here represent double-stranded oligonucleotides with compatible but non-identical overhanging ends on either both 5′- or both 3′- sides. In particular, small modules up to 50 bp can be generated simply by annealing synthetic oligonucleotides. In the example described below a two base-pair 3′-overhang was chosen which in the coding strand consists of GG, in the non-coding strand of CC. The non-identical nature of these overhangs allows the connection of the individual modular elements to each other in an orientation specific manner (FIG. 1).

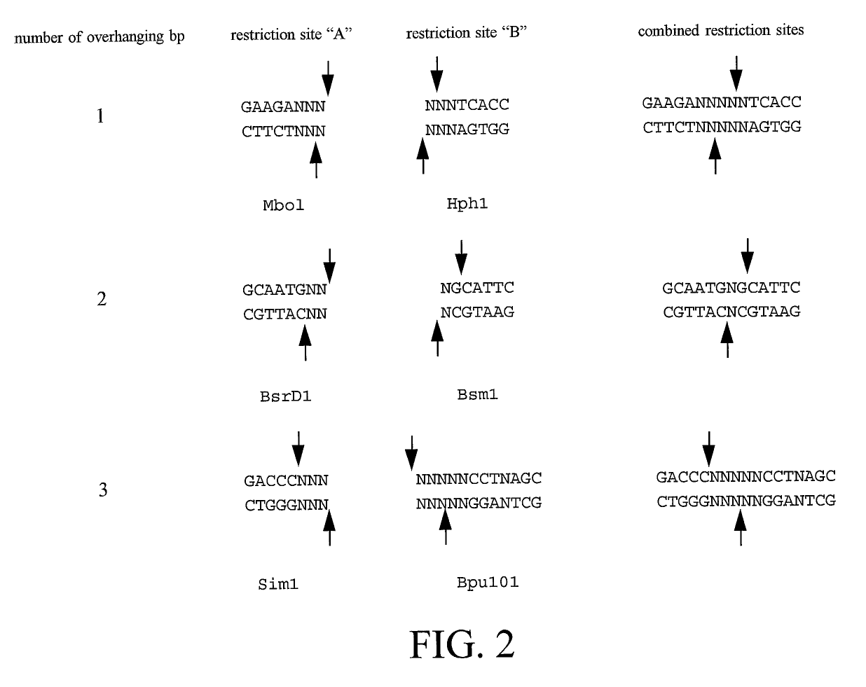

The ligation of these modules, shown in FIG. 1, can be performed by a polycondensation technique in which modules are directly connected to each other under controlled reaction conditions or with the help of cloning vectors. In order to insert these modules into vectors, a restriction site must be established which, after opening, produces the same overhangs for the vector as those chosen for the modular elements. This is achieved by introducing an interface into the poly-linker of the vector which at its center contains an interlocking pair of restriction sites (“A” and “B”, FIG. 2). The required features of these sites are: (i) both restriction sites produce either a 5′- or a 3′-overhang of the same length; (ii) the recognition sites are located outside of the cutting sites (or at least do not extend the overhang); (iii) the two recognition sites are located on each side of the cutting site, respectively; and (iv) the overhang produced after cutting on both strands is non-identical. In the following, examples are shown for compatible pairs of these restriction sites producing 1, 2 or 3 bp-overhangs, larger overhangs are also possible (FIG. 2).

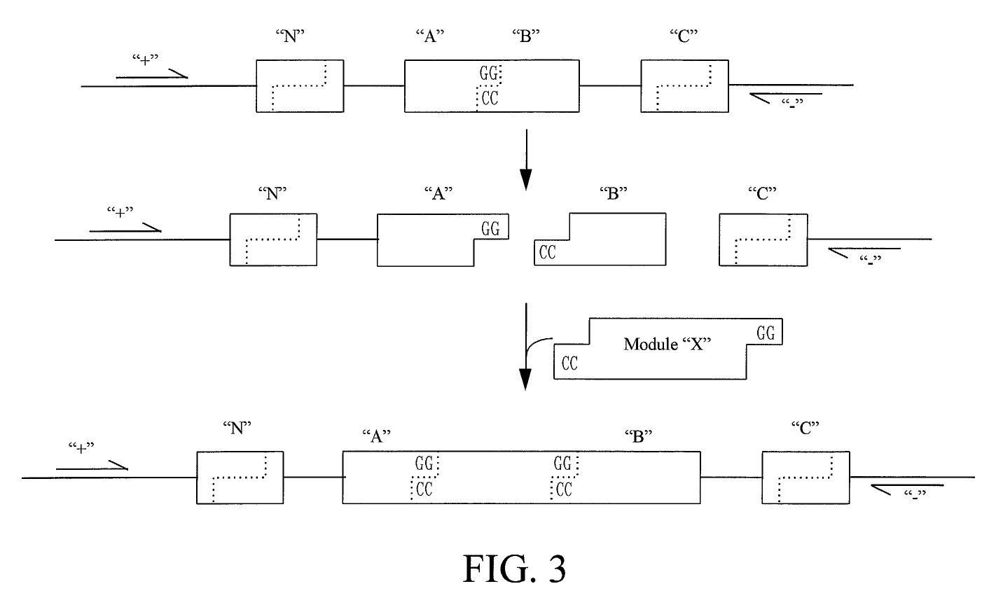

As shown in FIG. 2, vectors containing these combined sites can be opened by cutting with either “A” or “B”. Note that the bases at positions indicated by N can be freely selected. The choice of these bases at the cutting sites determines the sequence of the overhang and they are introduced into the vector with the interface oligonucleotide. In FIG. 3, an interface is used which produces the two base-pair GG/CC-overhang which matches to the overhang of the modules described earlier. The restriction sites “N” and “C” are standard sites of the poly-linker which are used to insert the interface oligonucleotide.

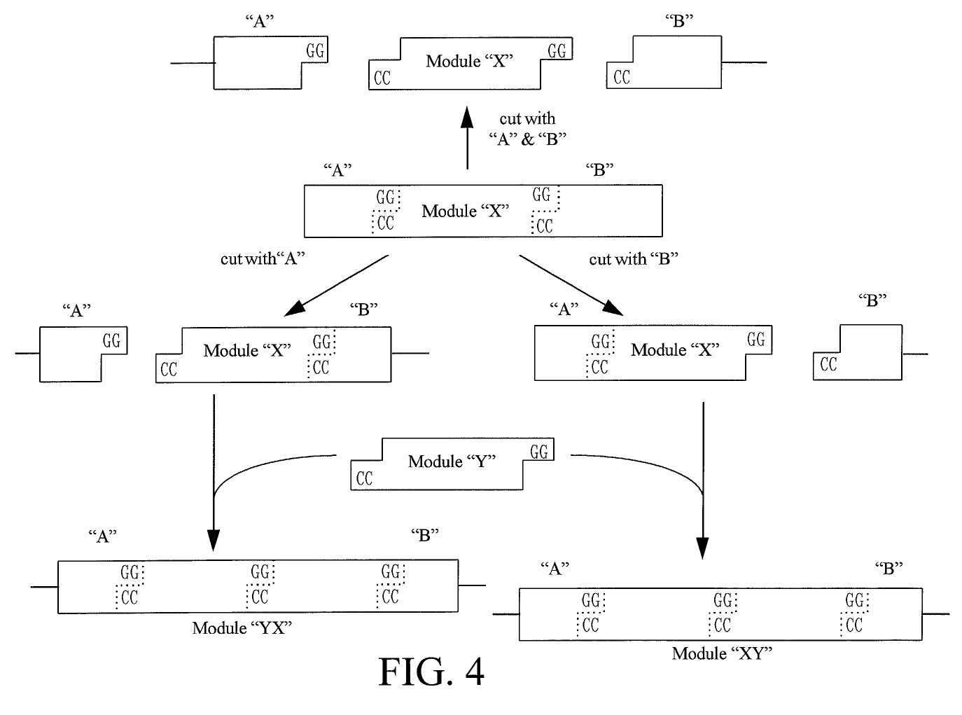

Amplification of the cloned module can be done by PCR utilizing the two PCR-priming sites provided by the vector (“+” and “−”, FIG. 3) or by transferring the construct into a host bacterium. The amplified module (shown in FIG. 4) can be released by using both restriction-enzymes “A” and “B” or the construct can be opened at one side for further extensions by cutting with either “A” or with “B”.

Depending on whether the expression-vector contains the interface or not, the full-length construct can finally be transferred into it by using either the sites “A” and “B”, or by releasing the entire construct by cutting with the standard enzymes “N” and “C”.

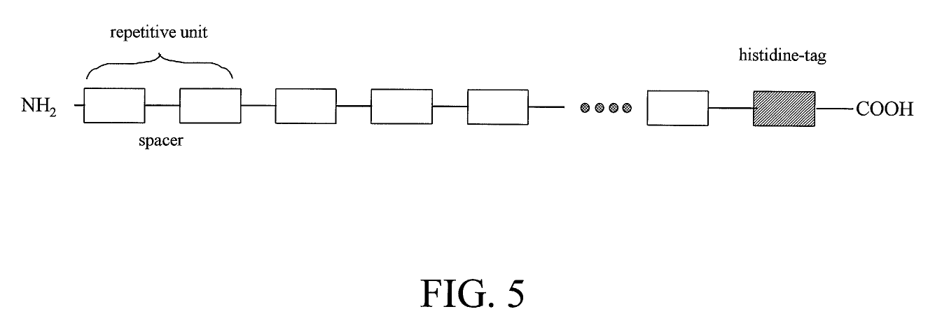

Using this method, large repetitive oligonucleotides can be constructed by carrying out several rounds of the poly-condensation steps, followed by either cloning into a vector for amplification or by successive extensions of a cloned module. A combination of both can also be employed. If larger, non-repetitive modules from a DNA-template (e.g., a gene) or pieces of DNA are to be connected to produce an oligonucleotide encoding a fusion protein, the non-identical overhangs can be formed with sites “A” or “B” (flanking the desired cutting site). This can be done by PCR using a primer which contains the recognition sequence at the necessary position, followed by restriction enzyme digestion. This method has already been used to construct repetitive oligonucleotides containing up to 32 T cell epitope-linker units which encode for proteins representing very effective T cell antigens (FIG. 5). It can, of course, also be used to generate other repetitive oligonucleotides, such as genes encoding for structural proteins (e.g., collagen or silk), gene-regulatory elements (e.g., repetitive enhancer-elements) or other non-repetitive DNA-constructs.

Construction and Expression of Oligonucleotides Encoding HA306-318 Oligomers

In the following, a procedure is described for the generation of artificial antigenic proteins whose primary structure consists almost entirely of tandem repeats of a T cell epitope-linker unit. In this example, the T cell epitopes are represented by HA306-318, a short amino acid sequence derived from the hemagglutinin protein of the influenza virus. Attached to a linker sequence (GGPGGGPGGGPGG), they form repetitive units of up to 32 copies which are directly connected to each other as shown in FIG. 5.

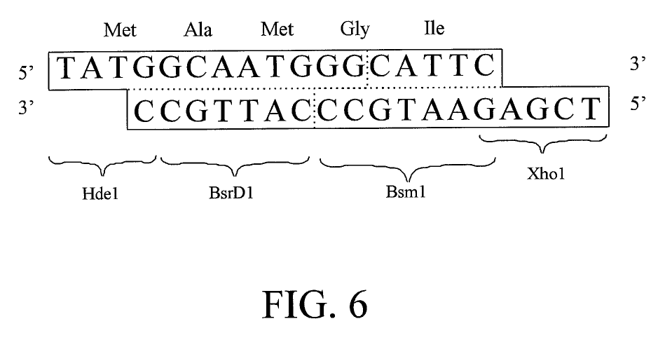

The proteins are produced through recombinant methodology using an E. coli expression system. The technique includes the addition of a C-terminal tag consisting of 6 histidine-residues, thus allowing the purification of the recombinant protein by affinity chromatography utilizing Ni-NTA-agarose (Quiagen). Two modified vectors are used for the construction (pCITE3a, Novagen) and expression (pET22b, Novagen) of the oligonucleotides encoding these proteins. Into both of these vectors, an interface-oligonucleotide was inserted which contained an interlocking pair of the non-palindromic restriction sites, BsrD I and Bsm I (FIG. 6).

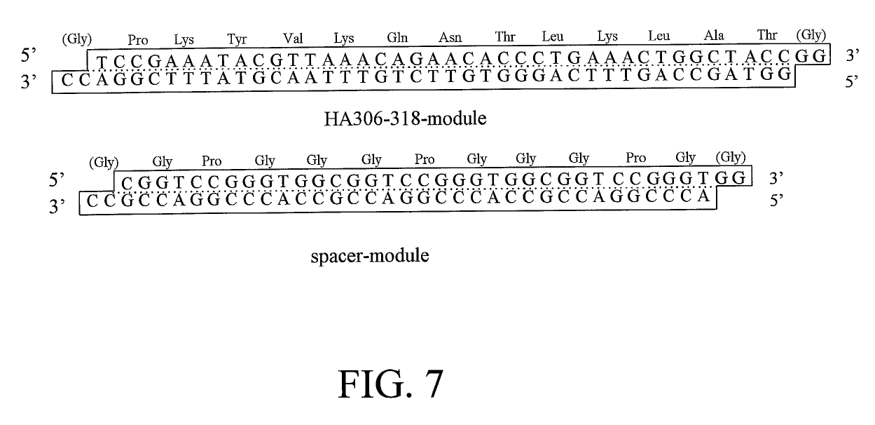

This interface was generated by annealing complementary strands of synthetic oligonucleotides and was inserted into the Nde I and Xho I sites of the poly-linker. The start codon is located within the Nde I site and leads to six histidine codons, located right behind the Xho I site which forms the histidine-tag (part of the pET22b-vector). The cloning site for the modules encoding the repetitive sequence is located at the glycine codon. The oligonucleotide is designed in such a way that enzyme digestion with either BsrD I or Bsm I produces a two bp 3′-overhang consisting of GG for the coding strand and of CC for the non-coding strand. All internal Bsm I and BsrD I sites of the pCITE3a vector had been previously removed by site-directed mutagenesis. FIG. 7 shows the two oligonucleotides required to construct the repetitive oligomer. These are the HA-306-318 module, which encodes the T cell epitope, and the spacer or linker module.

The modules can be generated by annealing complementary pairs of synthetic oligonucleotides. Their sequences are optimized in such a manner so that the codon-usage is selected based on their frequency in highly expressed genes in enterobacteria. Codons which are rarely found in these organisms are avoided, which assures efficient expression of the protein in the host bacterium. The sequence of the HA306-318 module (FIG. 7), therefore, is completely artificial and is not identical to the respective gene-region of the influenza hemagglutinin. Note that the overhanging ends of the modules are part of a glycine codon. This residue is actually part of the linker sequence, so that the repetitive HA306-318-linker unit does not contain any additional amino acids which result from its construction.

The HA306-318-linker unit is formed in the following way. After the modules are formed by annealing of the complementary strands and subsequent phosphorylation of their ends, each of these two modules is inserted into the modified pCITE vector. This is achieved by opening the interface using either Bsm I or BsrD I, followed by ligation of these modules using standard cloning protocols. Both DNA constructs are transformed into E. coli, isolated using a standard plasmid method, and finally the inserts are sequenced. The linker module is then amplified by PCR using a vector containing the linker sequence as a template and utilizing two priming sites located outside the interface in the vector. After gel purification of the PCR product, the linker module is released by a double-digest using BsrD I and Bsm I. This linker module is then inserted into a plasmid containing the correct HA306-318 module which has been opened at the C-terminal side by a Bsm I-digest. This construct, containing the monomeric HA306-318-linker unit, is transferred into bacteria, followed by isolation of the DNA. The dimeric unit is generated by cloning a HA306-318-linker module (amplified by PCR and digested with Bsm I and BsrD I as described above) back into a vector opened with either Bsm I or BsrD I which already contains one HA306-318-linker unit. A tetrameric unit is produced in the same way, by cloning a dimeric module back into a vector containing a dimeric insert.

In the next step, longer modules are formed by a polycondensation of tetrameric HA306-318-linker modules. Tetrameric modules are generated by PCR-amplification and Bsm I/BsrD I double-digest as described above. They are then directly connected to each other by a ligation carried out under controlled conditions using a high concentration of the module (16° C., 30′, appr. 10000 U/ml ligase). Under these conditions a series of modules are formed which contain 4 to more than 32 repetitive HA306-318-linker units. Separated by their size on a agarose gel, the reaction products yield a ladder in which the bands represent modules of increasing length. They are spaced by the size of one tetrameric unit (312 bp). These bands are then cut out of the gel, purified and finally cloned into the modified pET22b expression vector. Practically, modules containing up to 32 repetitive peptide-linker units can be isolated by this method. The DNA-amplification of these oligomer encoding nucleic acids in bacteria, as well as their expression, is carried out essentially according to standard procedures. However, highly repetitive DNA, as represented particularly by large HA306-318-linker oligomers, tends to be unstable, and elimination of part of the repetitive units can occur during the growth of the bacterial culture. Therefore, special care has to be taken in the choice of the strains used for these purposes. In our hands, best results were obtained by using E. coli TOP10 (Stratagene) for both large scale DNA production and protein expression. Since expression of genes cloned into the pET22b vector is driven by T7 RNA-polymerase (a viral enzyme which has to be provided by the host bacterium) the T7 RNA-polymerase gene has been previously transferred into the TOP10 strain. This was carried out be using a λDE3 lysogenization kit (Novagen). In addition, this strain was also transformed with the pLysS-plasmid (Novagen) ensuring a tight control of the T7 RNA-polymerase expression.

The expression of the oligomers is induced by adding IPTG (0.8 mM) to the culture at a density of about OD₆₀₀nm=0.6 and carried out for 4 h at 36° C. The bacteria are then harvested and lysed in a buffer containing 6 M guanidine/HCl, 500 mM NaCl, 100 mM Tris, 100 mM Na₂HPO₄, pH 8.0. The polypeptide oligomers are isolated by NTA-agarose (Quiagen) essentially according to the manufacturer's instructions utilizing the histidine-tag. In a final step, endotoxin (lipopolysaccharides) and other impurities are removed from the oligomers by separating the material on a reverse-phase C₄-HPLC-column (Vydac).

A size series of these materials was analyzed by SDS gel electrophoresis. Incubation of these peptide oligomers with empty HLA-DR1 molecules (produced using a baculovirus-based expression system) at 37° C. for 16 hours resulted in efficient loading of the HLA-DR1 molecules. Each oligomer yielded a ladder of bands, the number of bands increasing with the number of epitopes in the oligomer. This ladder is assumed to represent oligomers containing one HLA-DR molecule at the minimum with the larger bands representing more than one HLA-DR molecule per oligomer. Based on the fact that only a single major band is evident with the 4-mer (the polypeptide containing four linked HA306-318-linker subunits) and that a second prominent band appears for each 5-6 additional subunits (e.g., the 32-mer had 5 bands) additional HLA-DR subunits appear to be added at that spacing. A maximum of five bands is evident, presumably representing five HLA-DR molecules per oligomer. However, an anomaly in the gel migration is evident. First, using the 4-mer, the single major band observed at an apparent Mr of 45 kD (calculated size 57 kD: 11 kD oligomer+46 kD HLA-DR) runs faster than the HLA-DR/peptide complex itself (Mr 62 kD, calculated 47 kD). Moreover, the spacing between individual bands was equivalent to an addition of only about 32 kD per subunit, suggesting that the apparent Mr of each HLA-DR subunit was reduced by a conformational change upon oligomerization. In order to establish that HLA-DR itself was bound to the oligomers, and not separate α or β chains, Western blotting was carried out using polyclonal rabbit anti-αβ, anti-α and anti-β sera, clearly establishing that αβ HLA-DR heterodimers were bound to the oligomer.

Binding of MHC Binding Peptide Oligomers to APC Results in Signaling

The presumed oligomerization or clustering of HLA-DR upon interaction with T cell receptors on effector cells or with bivalent monoclonal antibodies results in intracellular signaling through proteins attached to the intracytoplasmic tail of HLA-DR. One consequence of the signaling is up regulation of expression of some surface molecules. The increase in expression of the three isotypes of class II molecules, HLA-DR, -DP, and -DQ, as well as of the cell adhesion molecule ICAM-1 were examined. No increase was observed on the addition of MHC binding peptide oligomer in the absence of gamma interferon, but the addition of gamma interferon (1-10 ng/ml) resulted in marked increases in surface expression, 5-10 fold greater than that seen in the presence of gamma interferon alone. The increase in HLA-DQ expression is especially notable since this isotype of class II MHC proteins is minimally inducible, if at all, on many cell types, including that used in the present study. A similar increase in the expression of ICAM-1 was observed and, together with enhanced expression of other cell surface molecules, may be responsible for the weak homotypic aggregation which was always seen in cultures of LG-2 cells. When staphylococcal enterotoxin A (SEA) was used to crosslink HLA-DR molecules, strong enhanced expression of ICAM-1 in the presence of 1-10 ng/ml of gamma interferon was also observed, but no changes were seen in the levels of expression of the three class II isotypes. No enhancement of B7.1 or B72.2 expression was observed with other oligomers or SEA under the conditions of these experiments.

Stimulation of HLA-DR1-Restricted T Cells by Monomers and Oligomers of HA306-318

Initially, an HA1.7 T cell clone that responds to HA306-318 presented by HLA-DR1 was used. For these experiments peripheral blood mononuclear cells (PBMC) from an HLA-DR1 donor were used as the antigen presenting cells (APC). The HA306-318 monomer/oligomer was either present continuously during the stimulation of HA1.7 at 37° C., or the APCs were pulsed for 15 minutes with the monomer/oligomer and, following extensive washing, used for the stimulation of T cells. When the monomer/oligomer was present continuously during the experiment, the oligomer was always more efficient as a stimulator than the monomer; in these experiments, increasing the size of the oligomer (4-mer to 32-mer) resulted in an enhancement of 10 fold (1 log) for the 4-mer to a maximum of 500-fold (almost 3 logs) for the 16-mer; enhancement diminished with larger oligomers. The enhancement relative to the hemagglutinin protein was still greater, amounting to more than 5 logs for the 16-mer. As expected, the addition of chloroquine (an agent that prevents acidification of endosomal vesicles) had no effect on antigen presentation (i.e., the oligomers do not require processing by acidic proteases in endosomal/lysosomal compartments for presentation).

A similar result was seen in a pulse experiment in which the monomer/oligomers were present for 15 minutes before being washed out, except that the enhancement was even larger, amounting to an increase of about 4 logs for the 16-mer relative to the monomer (but difficult to estimate exactly because under these conditions the peptide itself displayed very weak biological activity).

Priming of the Peripheral T Cell Response

For this dramatic enhancement of immunogenicity to be useful for vaccination in vivo, these oligomers must be capable of priming peripheral T cells. To examine this question, the T cells and APC in PBMC were used for priming. PBMC were stimulated and restimulated with monomers or oligomers at doses from 5 pg/ml-5 μg/ml, (i.e., over a 6 log range) in the presence of IL-2 over a period of 4 weeks. With monomers, the minimum dose for priming in this manner was 0.5 ng/ml. However, with the oligomers (e.g., the 12-mer or 16-mer) the minimum was 5 pg/ml, the minimum concentration actually tested (therefore, lower concentrations also may be effective). At “high” doses, 0.5 or 5 μg/ml, priming with monomer was still evident, but at 50 ng/ml or higher, no responses were seen with the 12-mer or 16-mer (i.e., this experiment appears to illustrate high zone tolerance) Thus the oligomers can be used in vivo both for immunization (priming of T cells) and for tolerization at high doses using either normal or altered peptide sequences as the epitopes. Moreover, lines derived from these primed T cells that had been generated in response to 5 or 50 pg/ml oligomer (12G7 and 16F1 respectively) responded to monomer, to oligomer, and to intact protein. Importantly, they also recognized virus-infected cells in an efficient manner. These facts establish the precondition for utilization of such materials for in vivo vaccination.

These data provide direct experimental support for the hypothesis that dimerization or oligomerization (clustering) of class II MHC molecules is an essential component of an effective immune response. Oligomers in which T cell epitopes are connected by amino acid linkers are superactivators of the immune response to a peptide derived from the influenza virus hemagglutinin. The resulting gain in sensitivity is extreme, amounting to 3-4 logs in comparison to monomer, or 4-5 logs in comparison to hemagglutinin protein. Surprisingly, the present experiment suggests that epitopes that bind class II MHC molecules are spaced by about 125-150 amino acids, (i.e., 5-6 epitope-linker subunits). The most effective oligomer in this set of experiments was a polymer of about 50,000 daltons containing 16 equally spaced HA306-318 epitopes, possibly allowing a maximum of 3 HLA-DR heterodimers in the cluster formed on incubation with soluble HLA-DR1. Smaller oligomers, the 4-mer and 8-mer capable of forming small amounts of clusters containing 2 HLA-DR subunits, did not activate efficiently. However, the detection of numbers of HLA-DR molecules added to the polymer was obtained with empty HLA-DR molecules in solution while the activation of cells was carried out by APC which contained HLA-DR molecules on cell surfaces, and only empty to the extent of 10-20 percent. Thus the nature of the cluster which provides effective activation of both naive T cells and previously primed T cell clones will require direct experimental investigation.

### Example 2

Production of Cross-Linked MHC Binding Peptide Dimers

Two basic types of cross-linked MHC binding peptides have been utilized. In these initial studies, the MHC binding peptides were cross-linked through their COOH ends because the distance between the two COOH ends is the shortest in the structure that has been revealed by crystallography. First, two peptides have been cross-linked using a linker of polyethyleneglycol (PEG) in which the average molecular weight is 800, where n (the number of repetitive ethylene glycol elements) averages 16 and the length is 30-40 Å. A variant of this basic structure has also been employed in which a β-alanine molecule (NH₂—CH₂—CH₂—COOH) has been added to each of the carboxy ends of the peptides in order to facilitate the condensation with PEG. Finally, a second type of linker has been employed in which sarcosine (N-methylglycine, CH₃—NH—CH₂—COOH) is the oligomerized subunit rather than ethyleneglycol. The two sarcosine elements are then connected by a single molecule of lysine through its ε and α amino groups. It will be recognized, however, that any diamine may be useful for this purpose.

Stimulation of HEL-Specific Hybridoma by Cross-Linked MHC Binding Peptide Dimers

Hen egg white lysozyme (HEL) is a standard protein antigen employed in experimental immunology. The immunodominant peptide has a core of amino acids 52-61 (HEL52-61). The peptides HEL48-61 and HEL48-63 have been frequently used in studies of the immune response to this protein antigen. Many hybridomas (immune T cells fused with a myeloma partner to allow ready growth in tissue culture) that respond to this peptide antigen have been prepared and are standard reagents in this field.

Using HEL48-63, to which the hybridomas respond more effectively than to HEL48-61, the response to the PEG-dimer was 1-2 logs more effective than that of the monomer HEL48-63. Moreover, an additional control in this experiment was HEL48-63 to which PEG was attached without addition of a second HEL48-63 peptide unit. The response to this control peptide was equal to or less than that to the monomer alone.

The β-Ala-PEG dimer was even more effective than the PEG dimer. With one hybridoma with a concentration of about 70 ng/ml, the enhancement of the PEG dimer relative to the monomer was at least 7 to 10 fold, while that of the β-Ala-PEG dimer was at least 25 to 30 fold. With a second hybridoma which responds at lower peptide concentrations, the response to 7 ng/ml of the PEG dimer was enhanced about 20 fold while that of the β-Ala-PEG dimer was enhanced 60 to 70 fold. The enhanced effectiveness of the β-Ala-PEG dimer was particularly evident at low concentrations. In particular, using the hybridoma 3A9.N49-11 at 5 ng/ml at 14 hours, virtually no response was seen to the monomer while a nearly maximum response (50 to 100 fold enhancement) was seen to the β-Ala-PEG dimer. It is important to emphasize that in immunizations in vivo only low concentrations of peptide antigens are obtained. These experiments illustrated the effectiveness of MHC binding peptide oligomers with two hybridomas, 3A9.9 and 3A9.N49-11, both of which express the same T cell receptor. That the phenomenon is not linked to the particular T-cell receptor is illustrated by an experiment in which two additional hybridomas, 1C5 and 4G4, were examined. The enhanced response to the β-Ala-PEG dimer is again clearly evident.

In order to extend these studies to human systems, similar experiments have been carried out with human T cells obtained from two individuals that had an immune response to influenza virus proteins. The β-Ala-PEG dimer in all cases provided an enhanced response.

In order to examine the effect of the nature of the linker (e.g., the polyethyleneglycol linker between two MHC binding peptide subunits of HEL 48-63), the binding peptide dimer was examined in which the two binding peptide units were cross-linked via a sarcosine linker with an average size 18 sarcosine elements plus one lysine. Enhanced proliferation was obtained with this material, again 1 to 2 logs.

### Example 3

Stimulation of Peripheral Blood T Cells in vitro

To determine whether MHC binding peptide oligomers are effective in triggering the immune response of peripheral blood T cells, an in vitro stimulation experiment was carried out, in which PBMCs (peripheral blood mononuclear cells) were exposed to the HA306-318 MHC binding peptide monomer or the HA 12-mer at antigen doses from 5 pg/ml-5 μg/ml (i.e., over a 6 log range). Each concentration was tested by preparing six independent wells (each containing approximately 100,000 PBMCs). After two rounds of stimulation with either HA306-318 monomer or the HA 12-mer, the specific proliferative response of these cultures was determined by challenging in vitro cultures with antigen-loaded target cells.

All T cell lines that were found to be specific for the HA306-318 epitope recognized both the monomer-pulsed targets as well as the targets pulsed with the 32-mer. None of them responded to only either one of the two antigens, indicating that T cell lines established by stimulation with oligomers also effectively recognize the monomeric peptide antigens and vice versa. The dose response pattern for the induction of in vitro cultures indicated that the HA 12-mer is at least 2 logs more effective than the peptide: with the peptide, the minimum dose required for establishing HA-specific T cell lines was found to be 0.5 ng/ml, while the 12-mer an efficient stimulation was still evident at 5 pg/ml, the lowest concentration used in these experiments.

These results demonstrate that the HA oligomers represent potent compounds to stimulate HA-specific T cells of the peripheral blood and promote their expansion. They also imply, that the enhanced immunogenicity is not just limited to some T cell clones but, rather, represents a general phenomenon which seems to apply to all HA306-318 specific T cells. The observed increase in sensitivity by 2-3 logs is reminiscent of the data obtained in stimulation experiments with the T cell clone HA1.7, and a similar dose response pattern was also found in several subsequent experiments using T cell lines established by this in vitro stimulation.

In vitro stimulation of PBMC also revealed another important feature of the HA oligomers: no specific response was detected for in vitro cultures stimulated with high doses of 12-mer. This finding seems to reflect high zone tolerance, a phenomenon which characterizes the lack of an antigen-specific T cell response after the exposure of the T cells to extremely high doses of peptide antigens. Mechanistically, high zone tolerance can be accomplished by two ways: by the induction of anergy, which refers to the transfer of the T cell into a state of non-responsiveness, or by the physical elimination of the reactive T cells. Both of these mechanisms are probably responsible for the 12-mer-induced high zone tolerance observed in in vitro cultures. On the one hand, a substantial fraction of a non-responsive CD4⁺ cells survives the oligomer treatment, which carry cell-surface markers of anergic cells (e.g., TCR^{low}, CD2^{high}, IL2R^{high}). The elimination, on the other hand, is particularly evident if established HA306-318-specific T cell lines are exposed to high oligomer concentrations. In one such experiment, the cell numbers of a T cell line originally stimulated with 5 ng/ml of the monomer (PD2) were monitored on days 4, 7, and 13 after the addition of the HA306-318 monomer or the HA 12-mer. Whereas incubation under these conditions with the monomer leads to an increase in the number of T cells, the PD2 cells treated with 5 or 50 82 g/ml 12-mer, show a drastic reduction in cell number by day 4. The difference between the stimulating effect of the peptide monomer and the tolerizing effect of the oligomer is particular evident on day 13: at that time point, even with the highest peptide concentration, the T cell population has expanded to approximately 6-7 times the original number, while the number of T cells treated with 5 or 50 μg/ml of the oligomer decreased to less than 10%. Control experiments demonstrated that this elimination is antigen-specific and MHC-restricted.

For in vitro stimulation, PBMC were isolated from the blood of an HLA-DR1-restricted donor by centrifugation on a Ficoll Paque cushion (Pharmacia). The cells were transferred into 96-well round-bottom plates and stimulated by the addition of titrated amounts of HA306-318 monomer or the HA 12-mer. For each concentration, the stimulation was carried out in 6 independent wells, each containing approximately 100,000 PBMCs. As a negative control, 12 wells were kept in which no antigen was added, in vitro cultures were maintained in RPMI medium supplemented with 5% autologous human serum following a standard protocol. On day 7, 5 U/ml IL2 (Boehringer) was added. The cultures were re-stimulated on day 12 by adding the same antigen concentration as used for the initial stimulation together with approximately 100,000 radiated autologous PBMCs (6000 rad). IL2 was added on day 15.

After an additional two weeks, the specific response against the HA306-318 epitope was tested in a proliferation assay. For that assay, 30 μl of each well of in vitro cultures was transferred into a 96-well round-bottom plate and incubated with approximately 25,000 radiated autologous EBV-transformed B cells (6000 rad). The EBV-transformed B cells had been previously pulsed for 30′ with 5,000 ng/ml HA306-318 monomer or HA 32-mer, or were incubated with no antigen. The experiment was carried out in RPMI 10% human serum (BioWhitaker). ³H-thymidine (5 μCi/ml) was added after 48 h and after an additional 24 h the assay was harvested and counted in a MicroBeta plate-reader (Wallac Oy, Finland).

The stimulation efficiency was determined by calculating the stimulation-index, which represents the ratio of the specific proliferative response triggered by antigen-loaded target cells and the non-specific response directed against the EBV-cell itself:

$$stimulation - index = \frac{cpm ( EBV pulsed with antigen )}{cpm ( EBV pulsed with no antigen )}$$

Several T cell lines obtained by this in vitro stimulation (e.g., PD2) were maintained and further expanded. For these lines, all subsequent rounds of re-stimulation were carried out every two weeks with approximately 25,000 radiated autologous EBV-transformed B cells/well previously pulsed for 2 h with 5 μg/ml HA306-318 monomer and approximately 100,000 radiated heterologous PBMC in RPMI 10% human serum (BioWhitaker). 5 U/ml IL2 (Boehringer) was added three days after re-stimulation.

For the proliferation assay, 96-well round-bottom plates were used with RPMI supplemented with 10% fetal calf serum (FCS). Radiated PBMCs were isolated as described above and used as target cells. Approximately 150,000 PBMCs/well were incubated for 2 h with titrated amounts of antigen at 37° C. before approximately 50,000 T cells/well were added. After 48 h 5 μCi/ml ³H-thymidine was added and after 72 h the assay was harvested and counted in a MicroBeta plate-reader (Wallac Oy, Finland).

For the high zone tolerance assay, approximately 50,000 T cells together with approximately 25,000 radiated autologous EBV-transformed B cells were incubated with titrated amounts of antigen in 96-well round-bottom plates in RPMI/10% human serum supplemented with 10 U/ml IL2. A 1:1 split was carried out on day 5 using RPMI/10% human serum, 10 U/ml IL2. The number of viable T cells was determined on the days 4, 7, and 13 by FACS analysis. For that determination, the cells were stained with propidium iodide (Boehringer) and the cell flow of an 80 μl aliquot of the culture was determined by the number of cells/minute passing a life-gate.

### Example 4

In Vitro Stimulation Using Various MHC Binding Peptide Oligomers

Experiments have been performed using four different MHC binding peptides (or “epitopes”) in MHC binding peptide oligomers. These MHC binding peptides are described in Table 1.

The HA306-318 epitope is an MHC binding peptide comprising residues 306-318 of the influenza virus hemagglutinin protein (strain X31). The HA306-318 epitope was chosen for analysis because (a) the epitope and its interactions with MHC class II molecules are well characterized (Stem et al. (1994), Nature 368:215-221, already of record); (b) it represents a promiscuous influenza virus-derived epitope, which has relevance as a T cell antigen during a viral infection; (c) several T cell lines and clones specific for this epitope are available; and (d) T cell cultures specific for HA306-318 epitope can usually be established relatively easily from the blood of donors with an appropriate MHC haplotype, and this allows the determination of the general potency of an antigen by directly challenging T cells isolated from the blood in an in vitro stimulation experiment. The HA306-318 epitope is known to bind to human MHC class II molecules such as DRB1*0101, DRB1*0401 or DRB5*0101.

The PLP139-151 (C140S) epitope is an MHC binding peptide derived from residues 139-151 of the mouse phospholipid protein, but in which the cysteine residue at position 140 was replaced with a serine residue (it is known that this modification does not generally effect recognition by specific T cells). The PLP139-151(C140S) epitope was chosen for analysis because it is known to be very effective in the induction of experimental autoimmune encephalitis (EAE), the mouse model system for multiple sclerosis (MS). Since the disease can be triggered by the injection of this peptide into certain mice strains (e.g. SJL), the PLP139-151(C140S) epitope is an excellent candidate epitope for the study of in vivo effects. PLP139-151 is known to bind to mouse MHC class II molecules of type H2-A^{s}.

The MBP86-100 epitope is an MHC binding peptide comprising residues 86-100 of the human myelin basic protein. The MBP86-100 epitope was chosen for analysis because of its association with multiple sclerosis (MS) and its ability to induce experimental autoimmune encephalitis (EAE) in certain mice strains. The MBP86-100 epitope represents a promiscuous epitope which is known to bind to a number of human and murine MHC class II molecules including HLA-DRB1*1501 and H2-A^{s}.

The CO260-272 epitope is an MHC binding peptide comprising residues 260-272 of the type II collagen protein, an epitope shared by several species, including human, mouse and bovine species. The CO260-272 epitope was chosen for analysis because type II collagen is known to be involved in the induction of rheumatoid arthritis (RA) and CO260-272 represents an immunodominant T cell epitope of this protein. In contrast to EAE, however, RA can only be induced by immunization with the protein and not with the peptide. The CO260-272 epitope is known to bind to the two human MHC class II molecules correlated with the development of RA, HLA-DRB1*0101 and HLA-DRB1*0401, and is also presented by the H2-A^{q} molecules of susceptible mouse strains (e.g., DBA/1).

Table 2, summarizes the results of in vitro studies performed, employing MHC binding peptide oligomers comprising 12 or 16 repeats of the HA306-318, PLP139-151 (C140S), MBP86-100, and CO260-272 MHC binding peptides (epitopes). In each case, an amino acid sequence of 13 residues (GGPGGGPGGGPGG) was used as a flexible molecular linker or “spacer sequence” between the epitopes.

Column 1 of Table 2 lists the MHC binding peptides/T cell epitopes which were incorporated into MHC binding peptide oligomers.

Column 2 of Table 2 shows the MHC restrictions which are associated with the MHC binding peptide in the assay employed, followed by the T cells or T cell hybridomas which were used and which were known to be reactive against the peptide, and the MHC-matched target cells to which the oligomers were expected to bind. The T cells were selected on the basis of their known reactivity against the monomers, the target cells were selected based on their known capability to act as antigen presenting cells (APCs) and based on the known expression of MHC class II molecules to match the restriction of the respective T cell.

The following cells were employed for the experiments presented in Table 2:

(a) T cells and T cell hybridomas: HA1.7 is an HLA-DRB1*0101-restricted human CD4⁺ T cell clone specific for the HA306-318 epitope; PD2 and 12G7 are HLA-DRB1*0101-restricted human CD4⁺ T cell lines specific for the HA306-318 epitope; 8475 and 8476 are HLA-DRB1*0401-restricted mouse CD4⁺ T cell hybridomas, which are specific for the HA306-318 epitope (generated in an HLA-DRB1*0401- transgenic mouse strain); SPL/1, SPL/2 and SPL/3 are H2-A^{s}-restricted mouse CD4⁺ T cell lines specific for the PLP139-151(C140S) epitope, SPL/2.2A8 is a T cell clone derived from the line SPL/2; hPLP/1 is an H2-A^{s}-restricted mouse T cell hybridoma specific for the PLP139-151(C140S) epitope (all PLP139-151(C140S)-reactive T cells were generated in SJL mice); OB.1A12 is an HLA-DRB1*1501-restricted human CD4⁺ T cell clone specific for the MBP86-100 epitope; M86P-1 is an H2-A^{s}-restricted mouse T cell line specific for the MBP86-100 epitope (generated in SJL mice); 2.15, 19.3, 3.19 and 5.1 are HLA-DRB1*0101-restricted mouse CD4⁺ T cell hybridomas reactive against the CO260-272 epitope (they were generated in an HLA-DRB 1*0101-transgenic mouse strain).

(b) The target cells in the in vitro experiments were the following: PBMC are peripheral blood mononuclear cells and were obtained from a healthy HLA-DRB1*0101-expressing human blood donor, and include a large fraction of natural APCs, such as B cells or dendritic cells; Priess (EBV) is an HLA-DRB1*0401-expressing human EBV-transformed B cell line; SJL spleen cells are cells isolated from the spleen of SJL mice, and include a large fraction of APCs, such as B cells and dendritic cells; and L57 cells are mouse fibroblast cells transfected with HLA-DRB 1*0101.

(c) PBMC used for in vitro-stimulation: In addition to antigen presenting cells, PBMC also contain a large fraction of CD4⁺ T cells. If non-irradiated PBMC are exposed to an antigen in vitro, it is possible to stimulate antigen-specific T cells, which are naturally occurring in the peripheral blood. For the in vitro stimulation experiment shown in Table 2, PBMC were used which were isolated from the blood of a healthy HLA-DRB1*0101-expressing donor.

Column 3 of Table 2 shows the type of assay which was performed to monitor the antigen-specific activation of the CD4⁺ T cells. Three different types of assays were employed:

(a) Proliferation assay. This assay was used in all experiments in which the response of T cell lines or T cell clones was tested. The proliferation assays were conducted essentially as described in Rötzschke et al. (1997), Proc. Natl. Acad. Sci. (USA) 94: 14642-14647). Briefly, the antigens (i.e., monomers or oligomers) were added to wells containing ˜50,000 T cells and irradiated target cells (e.g., ˜150,000 PBMC or ˜500,000 spleen cells) and proliferation was measured by determining the incorporation of ³H-thymidine added to the assays after 48 h-72 h for a period of 24 h. The proliferation of cells in response to the MHC binding peptide oligomers is specific for the T cells (irradiated target cells cannot proliferate) and is indicative of the efficiency of the antigen-specific stimulation.

(b) IL2 release assay. In experiments in which T cell hybridomas were used, antigen-specific stimulation was determined by an IL2 release assay (a proliferation assay cannot be employed because T cell hybridomas are immortalized cells, which proliferate constantly without the need for antigen-specific stimulation). The IL2 release assays were conducted essentially as described in Vignali et al. (1992), Int. Immunol. 4:621). Briefly, the antigen was added to wells containing ˜50,000 T cell hybridoma cells and target cells (e.g., ˜500,000 spleen cells, ˜25,000 EBV-transformed B cells, or ˜25,000 fibroblast cells). After an incubation of 24 h, the supernatants were collected and the amount of IL2 released was determined by addition to CTLL cells (a mouse T cell line which proliferates in response to IL2). IL2 release was measured by determining the proliferation of CTLL by the addition of ³H-thymidine to the culture. The amount of IL2 release by a T cell in response to MHC binding peptide oligomers is indicative of the efficiency of the antigen-specific stimulation of the T cell.

(c) in vitro stimulation. The in vitro stimulation of non-irradiated PBMC was carried out essentially as described for the generation of antigen-specific T cells by Wucherpfennig et al. (1994), J. Exp. Med. 179:279). Briefly, PBMC were isolated from the blood of a healthy donor and ˜100,000 PBMC/well were cultured in the presence of IL2 together with titrated amounts of either the peptide-antigen monomer or the respective oligomer. After 2 weeks, the primary cultures were re-stimulated by adding irradiated PBMCs (as APCs) together with the same amounts of antigen as used for the initial stimulation of the cultures. After an additional 2 weeks, the specificities of the individual cultures were tested by challenging the cells with peptide-loaded target cells (irradiated autologous EBV-transformed B cells pulsed for 2 h with 10 mg/ml peptide). To further demonstrate that the T cells generated by this in vitro stimulation have relevance for the defense of a viral infection, the individual cultures were also tested for their ability to recognize cells infected by the influenza virus. This was done by challenging the cells with autologous EBV-transformed B cells infected with influenza virus strain X31.

The right panel of Column 4 of Table 2 shows the number of repeats of the MHC binding peptide (epitope) in the oligomers employed. Column 5 of Table 2 shows the EC50_{(oligomer)} of the MHC binding peptide oligomers. EC50 is the effective concentration of the antigen (i.e., the MHC binding peptide oligomers or, as controls, MHC binding peptide monomers) needed to achieve 50% of the maximum effect. Column 6 of Table 2 shows the ratio of the EC50_{(peptide)}/EC50_{(oligomer)}. This ratio indicates the relative potency of the oligomer compared to the peptide monomer for the respective T cell/APC combination (a ratio greater than 1.0 indicates that the oligomer reaches half maximal effect at a lower concentration).

In each of the experiments listed in Table 2, the EC50s of the MHC binding peptide oligomers were less than or, at least, approximately equal to those of the monomers. This indicates that in each of these T cell/APC combinations, the MHC binding peptide oligomer retained the ability to trigger an antigen-specific T cell response.

For most of the T cell/APC combinations presented in Table 2, a ratio greater than 1.0 was determined. When used in vivo, MHC binding peptide oligomers will encounter a large variety of T cells. Therefore, a generally increased potency of the MHC binding peptide oligomers for the peptide-specific T cells and hybridomas in vitro suggests that they will also have generally increased potency in vivo (even if they exhibit decreased potency with respect to some individual T cell lines). The in vitro stimulation of HLA-DRB1*0101 expressing PBMC with the HA306-318 antigen closely resembles this in vivo situation. The outcome of this experiment was not biased by the expression of only one or a limited set of T cell receptors, since the antigen encounters the complete repertoire of antigen-specific T cells present in the peripheral blood. The T cell response of such an in vitro culture, therefore, reflects more adequately the potency that the monomer or the oligomer is likely to exhibit in vivo. In this experiment, a 100-fold to 1000-fold increase in potency of the 12-mer compared to the monomer was found.

Furthermore, all T cell cultures generated by the in vitro stimulation of PBMC efficiently recognized virus-infected cells. This finding is important with regard to a therapeutic or pharmaceutical application, since it indicates that T cells stimulated by MHC binding peptide oligomers derived from a viral epitope are reactive to cells infected by the virus.

The four different epitopes used in these experiments were selected because of their potential relevance for future applications and not because of any structural similarity to each other. For three out of the four epitopes (i.e., HA306-318, PLP139-151(C140S), and CO260-272), T cells recognized the oligomers more effectively than the monomers of the epitope. The increase in potency was substantial, indicated by EC50-concentrations up to 40-fold lower for the CO260-272 oligomer, up to 100-fold lower for the PLP139-151(C140S) oligomer, and up to 1000-fold lower for the HA306-318 oligomer. The fact that, for three out of the four unrelated antigens tested, the oligomerization of the epitope results in increased potency clearly indicates that this enhancement represents a general phenomenon. Furthermore, oligomers of the fourth epitope (i.e., MBP86-100) were recognized by T cells with a potency comparable to, or only slightly less than, the corresponding monomers, suggesting that the oligomers will have utility even if an increase in potency is not achieved.

### Example 5

In Vivo Tolerization Using HA306-318 and PLP139-151(C140S) Oligomers

In vivo studies were performed which further demonstrate that MHC binding peptide oligomers may reasonably be expected to have utility as pharmaceuticals or therapeutics. Experiments were conducted using the HA306-318 epitope and HLA-DRB1*0101-transgenic mice. In these experiments, mice were immunized (subcutaneously, with complete Freund's adjuvant (CFA) supplemented with 400 mg/mouse mycobacteria) with titrated amounts of monomer or 16-mer according to standard protocols (see, e.g., Greer et al. (1992), J. Immunol. 149:789). In the HA306-318 experiments, lymph node cells were removed on the 9th day after immunization and tested in vitro for their ability to recognize the HA306-318 peptide (upper panel). In the PLP139-151(C140S) system, the effectiveness of the immunization was monitored directly by the induction of experimental autoimmune encephalitis (EAE), since the epitope triggers the expansion of autoreactive T cells causing the disease. The severity of the disease was determined by scoring the symptoms of EAE according to a standard scale (see, e.g., Brocke et al. (1996), Nature 379:343-346).

In the HA306-318 experiments, an enhanced in vivo potency of the oligomer was evident in the lower amount of oligomer needed for the immunization of the mice to generate T cells specific for HA306-318. A shift of the response-curve towards lower concentrations was evident in the in vivo experiment, similar to that observed for the in vitro stimulation of PBMC. Antigen-specific T cells were generated with at least 10-fold more efficiency and could be generated by an immunization with only 50 ng, a concentration at which no specific response was detected for the lymph node cells isolated from mice immunized with the monomer. Furthermore, in the in vivo experiments with HA306-318, a lack of an antigen-specific T cell response was apparent after immunization of the mice with high concentrations of the oligomer (50 mg/mouse). This result was reminiscent of effects observed in the in vitro experiments and may suggest that this ‘high-zone tolerance’ is not limited to in vitro applications.

In the PLP139-151(C140S) experiments, the enhanced in vivo potency of the oligomerized epitope was evident in the lower amount of oligomer needed for the immunization of mice to induce the development of EAE. When equal amounts of monomer and oligomer were used, an accelerated and more drastic course of the autoimmune disease was observed for the mice injected with the oligomer. After an immunization with 50 mg of either the monomer or 16-mer, the onset of the disease is ˜3-4 days earlier with the 16-mer as compared to the monomer, and the disease is characterized by a very rapid progression. The course of the disease was almost as severe if only 10 mg of the oligomer was used. At this concentration, the monomer triggers only a very mild form of the disease.

These in vivo experiments indicate that both oligomers, the PLP139-151(C140S) 16-mer as well as of the HA306-318 16-mer, induce a T cell response which is antigen-specific and, in the case of the PLP139-151-system, results in the induction of symptoms expected after an immunization with the respective epitope. For the oligomers of both of these systems, a substantial enhancement of the in vivo potency was observed, which is consistent with the enhanced in vitro potency described above (which, in both cases, results in lower amounts of oligomer being needed to generate effects equivalent to that induced by the monomer).

It is known that the injection of high concentrations of T cell antigens, instead of leading to the activation of the immune system, often results in a tolerization of the test animal (e.g. Marusic et al. (1997), J. Exp. Med. 186:507; Liblau et al. (1996), Proc. Natl. Acad. Sci. (USA) 93:3031; Critchfield et al. (1994), Science 263:1139). The peptides used for the induction of tolerance are the same antigens which, at lower dosage, can cause T cell-stimulation. The main mechanism underlying this ‘high-zone’-tolerance seems to be the ‘suicidal’ elimination of T cells, since T cells recognizing excessively high densities of antigen are known to undergo programmed cell death (“apoptosis”) as a consequence of the over-stimulation (e.g. Critchfield et al. (1994), supra). In addition to the induction of apoptosis, “bystander-effects” can also play a role. For example, the additional recruitment of Th₂-cells has been found to result in a silencing of activated T cells and is probably caused by the release of interleukins such as IL4 (Brocke et al. (1996), Nature 379:343; Nicholson et al. (1995), Immunity 3:397)). Apparently, tolerization can be achieved either before or after the immune system encounters the antigen (e.g. Wu et al. (1997), J. Immunol. 159:3016). In most cases, however, successful tolerization has been reported if the tolerizing treatment is delivered prior to the stimulation of the T cells (e.g., Marusic et al. (1997), supra; Liblau et al. (1996), supra).

Experimental results have indicated that peptides representing T cell antigens can impose even stronger tolerizing effects if they are applied in an oligomerized form. To further corroborate this finding, two animal systems were utilized which allowed us to study the tolerizing effects of HA306-318 and PLP139-151(C140S) oligomers in vivo. The animals used for this purpose were mice of an HLA-DR1*0101-transgenic strain, and SJL mice expressing I-A^{s}, respectively. Since it is well known that both the dosage and route of administration of an antigen determines whether the antigen causes stimulation or tolerization in an animal, two different methods of administering the oligomers were used in these experiments. For stimulation of T cell response, the peptide antigen or oligomers were injected subcutaneously (s.c.), emulsified in Freund's adjuvant, supplemented with a ‘non-specifically’ stimulating reagent (mycobacyteria). For tolerization, in contrast, the peptides or the oligomers were delivered directly intravenously (i.v.), without any additional stimulating adjuvants or supplements. The goal of these experiments was to determine if the antigen-specific T cell response (induced by a subcutaneous (s.c.) application of the peptide antigen) could be effectively suppressed by an intravenous (i.v.) injection of the corresponding oligomer. Because these two antigen systems (i.e., HA306-318 and PLP139-151) exhibit different effects in vivo, two different experimental procedures were applied to determine the extent of the tolerization. In the case of the HA306-318-system, the presence or activity of HA306-318-specific T cells does not cause any obvious changes in the phenotype of the mice. Tolerance, therefore, had to be determined directly by testing the ability of the CD4⁺ T cells to respond to the HA306-318 antigen. This was done by an in vitro-proliferation assay using lymph node cells (LNCs) removed from the mice after they received the tolerizing and/or stimulating treatments. In this experiment, the successful induction of tolerance was indicated by a reduction of the antigen-specific proliferation of these LNCs compared to that of LNCs isolated from mice which received no i.v. injections of the antigen. In the case of the PLP139-151 system, it is known that the activation and expansion of PLP139-151-specific T cells leads to the induction of experimental autoimmune encephalitis (EAE). The tolerization of these auto-reactive T cells therefore effects the induction and progression of EAE, so that in this system it is not necessary to test the induction of tolerance on the cellular level. Rather, tolerization can be determined directly by scoring the clinical symptoms of EAE (e.g., Brocke et al. (1996), supra). A mean clinical score lower than that of the control group (which also received the disease-inducing s.c. injection with the peptide but not the i.v.-injections with oligomer of the invention) is therefore indicative of a successful tolerization of the animal. For these experiments the following protocols were used:

a) HA306-318. 50 ml of PBS (“control”), 50 mg HA306-318-peptide (“peptide”), or 50 mg of an HA306-318 oligomer with 16 repeats of the HA306-318 antigen joined by linkers (“16mer”), were dissolved in PBS (1 mg/ml), injected intravenously on day 0 and on day 3 into HLA-DR1*0101 transgenic B10.M-mice (two mice were used per group). On day 7 all mice received a subcutaneous injection of 25 mg HA306-318 peptide emulsified in Freund's adjuvant supplemented with 100 mg Mycobacteria tuberculosis. 8 days later the mice were sacrificed to remove the lymph nodes. LNCs were then cultured (500,000/well, cultured in DMEM-medium supplemented with 10% FCS) and challenged in a proliferation assay with titrated amounts of the HA306-318 peptide. The proliferative response of the LNCs was determined after 72 h by adding 1 mCi/well ³H-thymidine, harvesting the assay 24 h later.

b) PLP139-151(C140S). 50 mg of PLP139-151(C140S) peptide, emulsified in Freund's adjuvant supplemented with 400 mg Mycobacteria tuberculosis, were injected subcutaneously into SJL-mice. On day 1 and on day 2 200 ng of Pertussis toxin were injected intravenously (required for the acute induction of EAE; see, e.g., Greer et al. (1992), J. Immunol. 149:789). The mice were then split into three groups (each consisting of 3-5 mice), which received by intravenous injection on day 3 and on day 7 50 ml of PBS (“control”), or 50 mg of PLP139-151(C140S) peptide (“peptide”), or 50 mg of a PLP139-151(C140S) oligomer with 16 repeats of the PLP139-151(C140S) antigen joined by linkers (“16mer”), dissolved in PBS (1 mg/ml). Beginning on day 6, the mice were scored on a daily basis for the appearance of clinical signs of EAE.

The tolerization experiments with peptides and 16mers in the HA306-318 and the PLP139-151 systems produced the following results:

a) HA306-318-system. The peptide did not cause any significant tolerization, since the proliferative response of the LNCs isolated from mice treated with i.v.-injections of the peptide is equal to or, at best, only slightly weaker than that of the control. In contrast to this, the application of same amounts of the 16mer resulted in a very effective induction of tolerance. The LNCs from 16mer-treated mice show almost no response when challenged with the peptide antigen. Only some proliferation was still evident at the highest concentration, indicating that most of the T cells specific for the HA306-318-epitope had been effectively eliminated or silenced by the intravenous injection of the oligomer.

b) PLP139-151(C140S)-system Even more dramatic is the effect of the PLP139-151(C140S) oligomer. Here, the intravenous application of the 16mer causes a substantial suppression in the progression of EAE. The mean score of the mice treated with the oligomer never exceeds 1 and, by the end of the observation period, even goes down to 0, indicating a complete recovery of all mice treated with the 16mer. In contrast, the same dosage of peptide shows only a very weak, if any, effect: The onset of the disease is only slightly delayed (˜1 day) and the mean score, beginning from day 14, is practically identical to that of the control group. In contrast to the treatment with the oligomer, administration of the peptide could not stop the progression of the disease into a relatively severe form of EAE.

These results show that (i) both oligomers are, in fact, able to induce tolerance in vivo, and (ii) that both have a substantially higher potency in the induction of tolerance than the respective monomeric peptide. The oligomers of the invention effectively shut down the antigen-specific response at a dosage at which the peptide fails to show any substantial effect. In this context, it is also notable that in the EAE-model the tolerization was accomplished by an i.v. administration of the 16mer after the mice received the disease-inducing s.c. injection of the peptide. This timing is, of course, required for a therapeutic application, while most previous reports dealing with successful treatments of EAE used protocols in which the animals were tolerized prior to the disease-induction (e.g., Marusic et al. (1997), supra; Critchfield et al. (1994), supra).

The fact that for both systems tested, HA306-318 and PLP139-151 (C140S), effective tolerization in vivo was demonstrated suggests that it is a general feature of oligomerized T cell epitopes and that the higher potency, previously described for the T cell stimulation, is also effective for the tolerization. These data and, in particular, the successful prevention of the induction of EAE, the animal model system for multiple sclerosis, strongly suggest that oligomers can be expected to have a utility in pharmaceutical or therapeutic applications for the prevention or suppression of autoimmune diseases.

TABLE 1

| | Protein | | Biological |
|---|---|---|---|
| Epitope | source | MHC restriction | context |
| HA306-318 | Influenza | Human: | Human: |
| PKYVKQNTLKLAT | virus | DRB1*0101 | Influenza |
| | hemag- | DRB1*0401 | virus |
| | glutinin | DRB5*0101 | infection |
| | | (Promiscuous) | |
| | | Mouse: | |
| | | H2-E^{d} | |
| PLP139-151 (C140S) | Phospholipid | Mouse: | Mouse: |
| HSLGKWLGHPDKF | protein | H2-A^{s} | EAE |
| MBP86-100 | Myelin basic | Human: | Human: |
| | protein | | Multiple |
| | | | sclerosis |
| NPVVHFFKNIVTPRT | | HLA-DRB1*1501 | Mouse: |
| | | HLA-DRB5*0101 | EAE |
| | | (Promiscuous) | |
| | | Mouse: | |
| | | H2-A^{s} | |
| | | Rat: | |
| | | RT1.B^{L} | |
| C0260-272 | Type II | Human: | Human: |
| IAGFKGEQGPKGE | collagen | DRB1*0101 | Rheum- |
| | | DRB1*0401 | atoid |
| | | (Promiscuous) | arthritis |
| | | Mouse: | |
| | | H2-A^{q} | |

TABLE 2

| | Restriction | | | | | Ratio of |
|---|---|---|---|---|---|---|
| | T cell/ | | Oligomer | | | EC50_{(peptide)}/ |
| Epitope | Target cell | Assay | size | EC50_{(oligomer)} | | EC50_{(oligomer)} |
| HA306-318 | HLA-DRB1*0101 | Proliferation | 16-mer | ˜0.07 | ng/ml | ˜500 |
| | Clone HA1.7/ | | | | | |
| | PBMC | | | | | |
| | HLA-DRB1*0101 | Proliferation | 16-mer | ˜0.005 | ng/ml | ˜1000 |
| | Line PD2/ | | | | | |
| | PBMC | | | | | |
| | HLA-DRB1*0101 | Proliferation | 16-mer | ˜0.001 | ng/ml | ˜1000 |
| | Line 12G7/ | | | | | |
| | PBMC | | | | | |
| | HLA-DRB1*0101 | In vitro | 12-mer | ˜0.005 | ng/ml | ˜100-1000 |
| | in vitro stimulation of | stimulation | | | | |
| | PBMC | | | | | |
| | HLA-DRB1*0401 | IL2 release | 16-mer | ˜1.5 | ng/ml | ˜40 |
| | Mouse T cell | | | | | |
| | hybridoma 8475/ | | | | | |
| | Priess (EBV) | | | | | |
| | HLA-DRB1*0401 | IL2 release | 16-mer | ˜0.9 | ng/ml | ˜100 |
| | Mouse T cell | | | | | |
| | hybridoma 8476/ | | | | | |
| | Priess (EBV) | | | | | |
| PLP139-151 | H2-A^{s} | Proliferation | 16-mer | ˜1 | μg/ml | ˜5 |
| (C140S) | Mouse T cell line | | | | | |
| | SPL/1/ | | | | | |
| | SJL spleen cells | | | | | |
| | H2-A^{s} | Proliferation | 16-mer | ˜0.1 | μg/ml | ˜50 |
| | Mouse T cell line | | | | | |
| | SPL/2/ | | | | | |
| | SJL spleen cells | | | | | |
| | H2-A^{s} | Proliferation | 16-mer | ˜0.5 | μg/ml | ˜10 |
| | Mouse T cell line | | | | | |
| | SPL/3/ | | | | | |
| | SJL spleen cells | | | | | |
| | H2-A^{s} | Proliferation | 16-mer | ˜0.2 | μg/ml | ˜100 |
| | Mouse T cell clone | | | | | |
| | SPL/2.2A8/ | | | | | |
| | SJL spleen cells | | | | | |
| | H2-A^{s} | IL2 release | 16-mer | ˜1 | μg/ml | ˜10 |
| | Mouse T cell hybridoma | | | | | |
| | hPLP/1/ | | | | | |
| | SJL spleen cells | | | | | |
| MBP86-100 | HLA-DRB1*1501 | Proliferation | 16-mer | ˜0.1 | μg/ml | ˜0.7 |
| | T cell clone OB.1A12/ | | | | | |
| | PBMC | | | | | |
| | H2-A^{s} | Proliferation | 16-mer | ˜2 | μg/ml | ˜1 |
| | Mouse T cell line | | | | | |
| | M86P-1/ | | | | | |
| | SJL spleen cells | | | | | |
| CO260-272 | HLA-DRB1*0101 | IL2 release | 12-mer | ˜6 | μg/ml | ˜10-40 |
| | Mouse T cell hybridoma | | | | | |
| | 2.15/ | | | | | |
| | L57 (L-cells) | | | | | |
| | HLA-DRB1*0101 | IL2 release | 12-mer | ˜0.2 | μg/ml | ˜20 |
| | Mouse T cell hybridoma | | | | | |
| | 19.3/ | | | | | |
| | L57 (L-cells) | | | | | |
| | HLA-DRB1*0101 | IL2 release | 12-mer | ˜2 | μgm/ml | ˜0.5 |
| | Mouse T cell hybridoma | | | | | |
| | 3.19/ | | | | | |
| | L57 (L-cells) | | | | | |
| | HLA-DRB1*0101 | IL2 release | 12-mer | ˜1 | μg/ml | ˜1.8 |
| | Mouse T cell hybridoma | | | | | |
| | 5.1/ | | | | | |
| | L57 (L-cells) | | | | | |

We claim:

1. An MHC binding peptide oligomer comprising at least two MHC binding peptides covalently joined by a flexible molecular linker.

2. The oligomer of claim 1 wherein said MHC binding peptides are MHC class I binding peptides covalently joined C-terminus to C-terminus by a flexible molecular linker.

3. The oligomer of claim 2 wherein said flexible molecular linker is produced in vitro by chemical synthetic techniques.

4. The oligomer of claim 1 wherein said MHC binding peptides are MHC class II binding peptides.

5. The oligomer of claim 4 wherein said MHC class II binding peptides are covalently joined N-terminus to C-terminus.

6. The oligomer of claim 5 wherein said flexible molecular linker comprises naturally occurring amino acids.

7. The oligomer of claim 6 wherein said flexible molecular linker is produced in vivo by biosynthetic techniques.

8. An oligomer of claim 5 wherein said flexible molecular linker is produced in vitro by chemical synthetic techniques.

9. An oligomer of claim 4 wherein said MHC binding peptides are covalently joined C-terminus to C-terminus.

10. An oligomer of claim 9 which is produced in vitro by chemical synthetic techniques.

11. An oligomer of claim 4 wherein said MHC class binding peptides are covalently joined N-terminus to N-terminus.

12. An oligomer of claim 11 which is produced in vitro by chemical synthetic techniques.

13. Use of an MHC binding peptide oligomer in a composition for specifically activating a CD8⁺ T cell toward a cell presenting a predetermined antigenic peptide in association with a predetermined MHC class I molecule, wherein said specific activation is achieved by:
(a) providing an MHC binding peptide oligomer comprising at least two agonistic MHC class I binding peptides covalently joined by a flexible molecular linker, said MHC class I binding peptides corresponding to said predetermined antigenic peptide; and
(b) contacting, under physiological conditions, a cell bearing said MHC class I molecule on its cell surface with the oligomer of step (a).

14. A use as in claim 13 wherein said two MHC class I binding peptides are covalently joined C-terminus to C-terminus by a flexible molecular linker.

15. A use as in claim 16 wherein said flexible molecular linker is produced in vitro by chemical synthetic techniques.

16. Use of an MHC binding peptide oligomer in a composition for specifically inhibiting activation of a CD8⁺ T cell toward a cell presenting a predetermined antigenic peptide in association with a predetermined MHC class I molecule, wherein said specific inhibition is achieved by:
(a) providing an MHC binding peptide oligomer comprising at least two non-aginostic MHC class I binding peptides covalently joined C-terminus to C-terminus by a flexible molecular linker, said MHC class I binding peptides corresponding to said predetermined antigenic peptide; and
(b) contacting, under physiological conditions, a cell bearing said MHC class I molecules on its cell surface with the oligomer of step (a).

17. A use as in claim 16 wherein said non-agonistic peptide is selected from the group consisting of antagonistic, anergistic, blocking, tolerization-inducing, and apoptosis-inducing peptides.

18. A use as in claim 17 wherein said flexible molecular linker is produced in vitro by chemical synthetic techniques.

19. Use of an MHC binding peptide oligomer in a composition for activating a CD4⁺ T cell toward a cell presenting a predetermined antigenic peptide in association with a predetermined MHC class II molecule, wherein said activation is achieved by:
(a) providing an MHC binding peptide oligomer comprising at least two agonistic MHC class II binding peptides covalently joined by a flexible molecular linker, said MHC class II binding peptides comprising said predetermined antigenic peptide; and
(b) contacting, under physiological conditions, a cell bearing said MHC class II molecules on its cell surface with the oligomer of step (a).

20. A use as in claim 19 wherein said MHC binding peptides are covalently joined N-terminus to C-terminus.

21. A use as in claim 20 wherein said flexible molecular linker comprises naturally occurring amino acids.

22. A use as in claim 21 wherein said flexible molecular linker is produced in vivo by biosynthetic techniques.

23. A use as in claim 20 wherein said flexible molecular linker is produced in vitro by chemical synthetic techniques.

24. A use as in claim 19 wherein said MHC class II binding peptides are covalently joined C-terminus to C-terminus.

25. A use as in claim 24 wherein said flexible molecular linker is produced in vitro by chemical synthetic techniques.

26. A use as in claim 19 wherein said MHC binding peptides are covalently joined N-terminus to N-terminus.

27. A use as in claim 26 wherein said flexible molecular linker is produced in vitro by chemical synthetic techniques.

28. Use of an MHC binding peptide oligomer in a composition for specifically inhibiting activation of a CD4⁺ T cell toward a cell presenting a predetermined antigenic peptide in association with a predetermined MHC class II molecule, wherein said specific inhibition is achieved by:
(a) providing an MHC binding peptide oligomer comprising at least two non-agonistic MHC class II binding peptides covalently joined by a flexible molecular linker; and
(b) contacting, under physiological conditions, a cell bearing said MHC class II molecules on its cell surface with the oligomer of step (a).

29. A use as in claim 28 wherein said non-agonistic peptide is selected from the group consisting of antagonistic, anergistic, blocking, tolerization-inducing, and apoptosis-inducing peptides.

30. A use as in claim 29 wherein said MHC class II binding peptides are covalently joined N-terminus to C-terminus.

31. A use as in claim 30 wherein said flexible molecular linker comprises naturally occurring amino acids.

32. A use as in claim 31 wherein said flexible molecular linker is produced in vivo by biosynthetic techniques.

33. A use as in claim 30 wherein said flexible molecular linker is produced in vitro by chemical synthetic techniques.

34. A use as in claim 29 wherein said MHC class II binding peptides are covalently joined C-terminus to C-terminus.

35. A use as in claim 32 wherein said flexible molecular linker is produced in vitro by chemical synthetic techniques.

36. A use as in claim 29 wherein said MHC binding peptides are covalently joined N-terminus to N-terminus.

37. A use as in claim 20 wherein said flexible molecular linker is produced in vitro by chemical synthetic techniques.

38. Use of a DNA sequence encoding an MHC binding peptide oligomer in a medicament for genetic immunization against a predetermined pathogen, wherein said immunization is achieved by:
(a) providing said DNA sequence encoding an MHC binding peptide oligomer comprising at least two immunogenic MHC binding peptides derived from said pathogen covalently joined by a flexible molecular linker in an expression vector capable of replication and expression in mammalian cells; and
(b) introducing the expression vector of step (a) into the cells of an individual to be immunized.

39. Use of an MHC binding peptide oligomer in a medicament for eliminating tumor cells from an individual, wherein said elimination is achieved by:
(a) providing an MHC binding peptide oligomer comprising at least two agonistic, tumor-specific MHC binding peptides covalently joined by a flexible molecular linker; and
(b) contacting under physiological conditions, a cell bearing MHC molecules on its cell surface with the oligomer of step (a).

40. A use as in claim 39 wherein said MHC molecules are selected from the group consisting of MHC class I and MHC class II molecules.

41. Use of an MHC binding peptide oligomer in a composition for specifically inhibiting activation of a T cell by a predetermined antigenic peptide in association with a predetermined MHC molecule, wherein said specific inhibition is achieved by:
(a) providing an MHC binding peptide oligomer comprising at least two of said antigenic peptides covalently joined by a flexible molecular linker; and
(b) contacting, under physiological conditions, a cell bearing said MHC molecule on its cell surface with the oligomer of step (a) at a concentration sufficient for the induction of high zone tolerance.

42. A use as in claim 41 wherein said T cell is CD4⁺.

43. A use as in claim 41 wherein said T cell is CD8⁺.

44. Use of an MHC binding peptide oligomer for producing an immunomodulatory composition, comprising:
(a) identifying an MHC binding peptide; and
(b) preparing an MHC binding peptide oligomer comprising at least two copies of the MHC binding peptide of step (a) covalently joined by a flexible molecular linker.

45. A use as in any one of claims 13-44 wherein said flexible molecular linker has a backbone length of at least about 50-80 Å.

46. A use as in any one of claims 13-44 wherein said flexible molecular linker has a backbone length of at least about 540 Å.

47. A use as in any one of claims 13-44 wherein said flexible molecular linker comprises at least about 10-20 amino acid residues.

48. A use as in any one of claims 13-44 wherein said flexible molecular linker comprises at least about 125 amino acid residues.

49. A use as in any one of claims 13-20, 23-30, or 33-37, 39-44, wherein said flexible molecular linker comprises a polymer or copolymer of organic acids, aldehydes, alcohols, thiols, or amines.

50. A use as in any one of claims 13-20, 23-30, or 33-37, 39-44, wherein said flexible molecular linker comprises a polymer or copolymer of hydroxy-, amino-, or di-carboxylic acids.

51. A use as in any one of claims 13-20, 23-30, or 33-37, 39-44, wherein said flexible molecular linker comprises a polymer or copolymer of glycolic acid, lactic acid, sebacic acid, or sarcosine.

52. A use as in any one of claims 13-20, 23-30, or 33-37, 39-44, wherein said flexible molecular linker comprises a polymer or copolymer of saturated or unsaturated hydrocarbons.

53. A use as in any one of claims 13-20, 23-30, or 33-37, 39-44, wherein said flexible molecular linker comprises a polymer or copolymer of ethylene glycol, propylene glycol, or saccharides.

54. A use as in any one of claims 13-20, 23-30, or 33-37, 39-44, wherein said flexible molecular linker comprises a polymer of polyethylene glycol and β-alanine or lysine.

55. A use as in any one of claims 13-20, 23-30, or 33-37, 39-44, wherein said flexible molecular linker comprises a polymer or copolymer of non-naturally occurring amino acids.

56. A use as in any one of claims 13-20, 23-30, or 33-37, 39-44, wherein said flexible molecular linker comprises a polymer of sarcosine and lysine or β-alanine.

57. A use as in any one of claims 13-56 wherein said MHC binding peptide comprises a human autoantigenic peptide derived from a protein selected from the group consisting of myelin basic protein (MBP), proteolipid protein (PLP), AChRα, collagen type II, HSP70, and glutamic acid decarboxylase

58. A use as in any one of claims 13-56 wherein said MHC binding peptide oligomer comprises at least four MHC binding peptides.

59. A use as in any one of claims 13-56 wherein said MHC binding peptide oligomer comprises at least eight MHC binding peptides.

60. A use as in any one of claims 13-56 wherein said MHC binding peptide oligomer comprises at least sixteen MHC binding peptides.

61. An oligomer as in any one of claims 1-12, wherein said flexible molecular linker has a backbone length of at least about 50-80 Å.

62. An oligomer as in any one of claims 1-12, wherein said flexible molecular linker has a backbone length of at least about 540 Å.

63. An oligomer as in any one of claims 1-12, wherein said flexible molecular linker comprises at least about 10-20 amino acid residues.

64. An oligomer as in any one of claims 1-12, wherein said flexible molecular linker comprises at least about 125 amino acid residues.

65. An oligomer as in any one of claims 1-12, wherein said flexible molecular linker comprises a polymer or copolymer of organic acids, aldehydes, alcohols, thiols, or amines.

66. An oligomer as in any one of claims 1-12, wherein said flexible molecular linker comprises a polymer or copolymer of hydroxy-, amino-, or di-carboxylic acids.

67. An oligomer as in any one of claims 1-12, wherein said flexible molecular linker comprises a polymer or copolymer of glycolic acid, lactic acid, sebacic acid, or sarcosine.

68. An oligomer as in any one of claims 1-12, wherein said flexible molecular linker comprises a polymer or copolymer of saturated or unsaturated hydrocarbons.

69. An oligomer as in any one of claims 1-12, wherein said flexible molecular linker comprises a polymer or copolymer of ethylene glycol, propylene glycol, or saccharides.

70. An oligomer as in any one of claims 1-12, wherein said flexible molecular linker comprises a polymer of polyethylene glycol and β-alanine or lysine.

71. An oligomer as in any one of claims 1-12, wherein said flexible molecular linker comprises a polymer or copolymer of non-naturally occurring amino acids.

72. An oligomer as in any one of claims 1-12, wherein said flexible molecular linker comprises a polymer of sarcosine and lysine or β-alanine.

73. An oligomer as in any one of claims 1-12, wherein said MHC binding peptide comprises a human autoreactive peptide selected from the group consisting of myelin basic protein (MBP), proteolipid protein (PLP), AChRα, collagen type II, HSP70, and glutamic acid decarboxylase

74. On oligomer as in any one of claims 1-12, wherein said MHC binding peptide oligomer comprises at least four MHC binding peptides.

75. An oligomer as in any one of claims 1-12, wherein said MHC binding peptide oligomer comprises at least eight MHC binding peptides.

76. A MHC oligomer as in any one of claims 1-12, said MHC binding peptide oligomer comprises at least sixteen MHC binding peptides.

77. A use as in claim 57 wherein said MHC binding peptide comprises an amino acid sequence selected from the group consisting of
residues 57-75, residues 81-99, residues 82-100, residues 83-97, residues 83-99, residues 84-102, residues 87-99, residues 87-106, residues 131-145, residues 131-159, residues 139-153, residues 143-168, residues 148-162, residues 149-162, and residues 154-172 of myelin basic protein (MBP).

78. A use as in claim 57 wherein said MHC binding peptide comprises an amino acid sequence comprising amino acid residues selected from the group consisting of residues 40-60, residues 89-106, and residues 139-151, of proteolipid protein (PLP).

79. A use as in claim 57 wherein said MHC binding peptide comprises an amino acid sequence comprising amino acid residues selected from the group consisting of
residues 48-67, residues 55-63, residues 144-163, residues 146-162, residues 195-212, residues 214-234, residues 259-271, residues 311-318, and residues 387-405, of AChRα.

80. A use as in claim 57 wherein said MHC binding peptide comprises an amino acid sequence comprising amino acid residues selected from the group consisting of
residues 258-260, residues 258-270, residues 259-273, residues 260-267, residues 261-273, residues 262-270 and residues 263-270 of collagen type II.

81. A use as in claim 57 wherein said MHC binding peptide comprises an amino acid sequence comprising amino acid residues of HSP70 selected from the group consisting of
residues 150-169, residues 221-240 and residues 261-280 of the of HSP70 protein of M. tuberculum.

82. A use as in claim 57 wherein said MHC binding peptide comprises an amino acid sequence comprising amino acid residues selected from the group consisting of
residues 78-97, residues 202-211, residues 217-236, and residues 247-279 of glutamic acid decarboxylase.

83. A use as in claim 73 wherein said MHC binding peptide comprises an amino acid sequence comprising amino acid residues selected from the group consisting of
residues 57-75, residues 81-99, residues 82-100, residues 83-97, residues 83-99, residues 84-102, residues 87-99, residues 87-106, residues 131-145, residues 131-159, residues 139-153, residues 143-168, residues 148-162, residues 149-162, and residues 154-172 of myelin basic protein (MBP).

84. A use as in claim 73 wherein said MHC binding peptide comprises an amino acid sequence comprising amino acid residues selected from the group consisting of
residues 40-60, residues 89-106, and residues 139-151, of proteolipid protein (PLP).

85. A use as in claim 73 wherein said MHC binding peptide comprises an amino acid sequence comprising amino acid residues selected from the group consisting of
residues 48-67, residues 55-63, residues 144-163, residues 146-162, residues 195-212, residues 214-234, residues 259-271, residues 311-318, and residues 387-405, of AChRα.

86. A use as in claim 73 wherein said MHC binding peptide comprises an amino acid sequence comprising amino acid residues selected from the group consisting of
residues 258-260, residues 258-270, residues 259-273, residues 260-267, residues 261-273, residues 262-270 and residues 263-270 of collagen type II.

87. A use as in claim 73 wherein said MHC binding peptide comprises an amino acid sequence comprising amino acid residues selected from the group consisting of
residues 150-169, residues 221-240 and residues 261-280 of the of HSP70 protein of M. tuberculum.

88. A use as in claim 73 wherein said MHC binding peptide comprises an amino acid sequence comprising amino acid residues selected from the group consisting of
residues 78-97, residues 202-211, residues 217-236, and residues 247-279 of glutamic acid decarboxylase
